(19) United States
(12) Patent Application Publication
(10) Pub. No.: US 2020/0268795 A1
(43) Pub. Date: Aug. 27, 2020

(54) INTERFERON-GAMMA ATTENUATES ANTI-TUMOR IMMUNE RESPONSE TO CHECKPOINT BLOCKADE

(71) Applicant: The Regents of the University of California, Oakland, CA (US)

(72) Inventors: Chien-Chun Steven PAI, Oakland, CA (US); Lawrence FONG, Oakland, CA (US)

(21) Appl. No.: 16/646,512

(22) PCT Filed: Sep. 24, 2018
(86) PCT No.: PCT/US2018/052416

§ 371 Date: Mar. 11, 2020

Related U.S. Application Data
(60) Provisional application No. 62/561,849, filed on Sep. 22, 2017.

(51) Int. Cl. A61K 35/17; C12N 5/0783; C12N 15/113; C07K 16/28; A61K 39/395; A61K 45/06; A61P 35/00

(52) U.S. Cl. CPC A61K 35/17; C12N 5/0636; C12N 15/1138; C07K 16/2809; C07K 16/2887; A61K 39/3955; C12N 2510/00; A61P 35/00; C07K 2317/31; C07K 2317/24; C07K 2319/03; C07K 2319/30; A61K 45/06

(57) ABSTRACT

T-cells that does not express a functional Interferon-γ (IFN-γ) receptor are provided as well as methods of treating cancers with the T-cells, optionally in combination with immune checkpoint pathway inhibitors.

### CROSS-REFERENCE TO RELATED PATENT APPLICATIONS

The present application claims benefit of priority to U.S. Provisional Patent Application No. 62/561,849, filed Sep. 22, 2017, which is incorporated by reference for all purposes.

### BACKGROUND OF THE INVENTION

In recent years, immune checkpoint inhibitors are being rapidly approved for the management of advanced malignancies, including melanoma, non-small cell lung cancer, renal cell carcinoma, urothelial carcinoma, and head and neck cancer (M. K. Callahan et al., Immunity 44, 1069-1078 (2016)). However, only a small subset (10-30%) of patients responds to single agent immune checkpoint therapy (C. Robert et al., The New England journal of medicine 372, 2521-2532 (2015)), and a myriad of combination strategies are currently being actively investigated in clinical trials to enhance the potency of this approach.

Co-targeting of CTLA-4 and PD-1 immune checkpoint pathways is one strategy that has demonstrated significant clinical outcomes in melanoma (J. Larkin et al., The New England journal of medicine 373, 23-34 (2015)). Encouraging data combining ipilimumab and nivolumab has also been reported for non-small cell lung cancer (M. D. Hellmann et al., The lancet oncology 18, 31-41 (2017)). Despite these advances, a significant fraction of patients still does not achieve objective responses to checkpoint inhibitors. In multiple large randomized trials (E. D. Kwon et al., The lancet oncology 15, 700-712 (2014); H. Borghaei et al., The New England journal of medicine 373, 1627-1639 (2015); J. Bellmunt et al., The New England journal of medicine 376, 1015-1026 (2017)), patients receiving immune checkpoint inhibitors actually have worse survival outcomes compared to control arms during the initial months of treatment, at a time before immune-related toxicities fully manifest. In addition, the phenomena of “tumor hyper-progression” has been recently described where 9% of cancer patients receiving immune checkpoint inhibitors have accelerated tumor growth (S. Champiat et al., Clinical cancer research: an official journal of the American Association for Cancer Research 23, 1920-1928 (2017)).

Several studies have contributed to the understanding of mechanisms underlying differential responses and mechanisms of resistance to immune checkpoint strategies. This includes the role of pre-existing CD8+ T cells in the tumor invasive margins in melanoma patients treated with PD-1 blockade (P. C. Tumeh et al., Nature 515, 568-571 (2014)), and interferon-dependent expression of inhibitory ligands on tumor cells that mediate therapy resistance (J. L. Benci et al., Cell 167, 1540-1554 e1512 (2016)). However, the influence of tumor burden on the efficacy of immune checkpoint therapies has not been carefully investigated.

### BRIEF SUMMARY OF THE INVENTION

In some aspects, a human tumor-specific T-cell is provided that does not express a functional Interferon-γ (IFN-γ) receptor. In some embodiments, the T-cell comprises a mutation compared to wildtype that blocks IFN-γ receptor expression. In some embodiments, the mutation is a mutation in an IFN-γ receptor promoter or IFN-γ receptor coding sequence. In some embodiments, part or all of a coding sequence for IFN-γ receptor has been deleted. In some embodiments, the T-cell comprises an siRNA or antisense polynucleotide that inhibits expression of IFN-γ receptor. In some embodiments, the T-cell comprises a tumor-specific T-cell receptor. In some embodiments, the tumor-specific T-cell receptor is heterologous to the T-cell. In some embodiments, the T-cell is bound by a bispecific binding reagent that binds CD-3 and a tumor antigen. In some embodiments, the tumor antigen is CD-20. In some embodiments, the bispecific binding reagent is a bispecific antibody. In some embodiments, the T-cell comprises a heterologous chimeric antigen receptor (CAR).

In some aspects, a method of killing cancer cells in a human is provided. In some embodiments, the method comprises, administering to the human a sufficient number of the T-cell as described above o elsewhere herein to kill cancer cells in the human. In some embodiments, the method further comprises administering to the human (one or two or more immune pathway inhibitors (e.g., a CTL-4 inhibitor and a PD-1 inhibitor). In some embodiments, the T-cells have been obtained from the human and then altered to inhibit expression of the functional Interferon-γ (IFN-γ) receptor. In some embodiments, the human has melanoma. In some embodiments, the method further comprises administering to the human an antibody that binds to IFN-γ (e.g., a sufficient amount of an antibody that binds to IFN-γ to promote survival of the administered T-cells).

Also provided is a method of killing cancer cells in a human, the method comprising, administering to the human an effective amount of a JAK inhibitor, a CTL-4 inhibitor and a PD-1 inhibitor, and optionally a further agent that is toxic to cancer cells, thereby killing cancer cells in the human. In some embodiments, the human has melanoma. In some embodiments, the method further comprises administering to the human a sufficient number of the T-cell as described above or elsewhere herein.

### BRIEF DESCRIPTION OF THE DRAWINGS

FIGS. 1A-L. Combination checkpoint blockade enhances anti-tumor responses against established tumors. C57BL/6j mice were implanted with different types of tumors (1×10⁶ cells/mouse) and treated with checkpoint inhibitors as indicated. (A) Schema of mice injected with MC-38 and treated with checkpoint blockade. (B) Schema of TRAMP-C2 bearing mice treated with checkpoint blockade. (C) The tumor growth curve in MC-38 model. (D) The tumor growth curve in TRAMP-C2 model. (E) Representative H&E staining from tumor samples harvested three days after the last treatment. (F) Flow staining of CD45+CD3+CD4+Foxp3+ T cells within tumor microenvironment. (G) Percentage of CD4+Foxp3+ cells among CD45+ (H) Ratio of total numbers of CD8+ T cells over Treg cells (I-J) Flow gating strategy of CD8+ T cell subsets in tumors. Tim-3 expression on CD8 subsets isolated from tumor tissues (K-L) Tetramer staining among tumor infiltrating CD8+ T cell populations. Cells were pre-gated on CD45+CD3+CD8+. Data were collected from at least 8 mice per group with two independent experiments. Statistical differences were calculated by one-way or two-way ANOVA with post-hoc Tukey test. (*)P<0.05, (**)P<0.01, (****)P<0.0001.

FIGS. 2A-H. Opposing effect of combination checkpoint blockade with low tumor burden. (A) Schema of early intervention versus therapeutic treatment with dual checkpoint blockade prior to the development of palpable tumors. (B) TRAMP-C2 tumor growth with early intervention in different treatment groups. The dose for each checkpoint inhibitor is 10 mg/kg. (C) Tumor growth with two different doses of anti-PD-1 DANA antibody combined with anti-CTLA-4 blockade. The doses of anti-PD-1 DANA are 2.5 mg/kg and 10 mg/kg. (D) Different clone of anti-PD1 antibody in anti-tumor effects. The dose for each checkpoint inhibitor is 10 mg/kg. (E) Tumor growth curves of combination of CTLA-4 blockade with GVAX vaccine. The dose for anti-CTLA-4 is 10 mg/kg. The dose for GVAX vaccine is 106 irradiated cells. (F-H) 152 metastatic melanoma patients treated with checkpoint inhibitors were stratified into three groups with different ranges of baseline tumor size as measured by radiographic imaging. The best overall response rate (RECIST 1.1) in patients treated with monotherapy or combination therapy are presented for each stratum. Animal studies in figure B-D were from two independent experiments with at least 10 mice per group. FIG. 2E was from one animal study with 10 mice per group. Patient data were collected from 152 patients in clinical trials. Statistical analyses were calculated by two-way ANOVA with post-hoc test or two-side Mann-Whitney test. (*)P<0.05, (**)P<0.01, (***)P<0.001, (****)P<0.0001.

FIGS. 3A-I. Dynamics of tumor-specific T cells post checkpoint blockade. Mice implanted with TRAMP-C2 tumors were treated with checkpoint inhibitors on day 3, 6, and 9. Spleens were harvested on day 11 and day 28. (A) Schema of animal studies. (B) Flow gating of antigen specific CD45+CD3+CD8+ T cells against the immunodominant Spas-1 epitope and minor Spas-2 epitope. (C) Total CD8+Spas-1 T cells isolated at day 11. (D) Total CD8+Spas-1 T cells at day 28. (E) Dynamic changes of CD8+Spas-1 T cells over time. (F) Total CD8+Spas-2 T cells at day 11. (G) Total CD8+Spas-2 T cells at day 28. (H) Dynamic changes of CD8+Spas-2 T cells over time. Data were from 2-3 independent experiments with 9-12 mice per groups. (I) MART-1/MHC multimer staining over time in a metastatic melanoma patient treated with combination anti-PD-1 and anti-CTLA-4 treatment. Statistical analyses were calculated by one-way ANOVA with post-hoc Tukey test. (*)P<0.05, (**)P<0.01.

FIGS. 4A-G. Tumor-specific T cell loss with combination therapy is associated with IFN-γ. CD8+Spas-1 T cells were sorted from draining lymph nodes on day 28 after checkpoint inhibitor treatments. (A) FACS gating of CD8+Spas-1 T cell subset. (B) gene expression of pro-apoptotic gene clusters by cDNA microarray (C) Gene expression for caspase family (D) Gene expression for anti-apoptotic transcripts. (E) Active caspase-3 expression among CD8+Spas-1 T cell was determined by flow cytometry. (F) Percentage of active caspase-3 expression among Spas-1-reactive CD8+ T cells. (G) Mice implanted with TRAMP-C2 were treated with checkpoint inhibitors on day 3, 6, and 9. Serum cytokine levels were detected on day 11. Data were collected from 5 mice per group in FIGS. 4A-D and G. FIGS. 4E-F were representative from two independent experiments with 10 mice per group. Statistical analyses were calculated by one-way ANOVA with post-hoc Tukey test. (*)P<0.05, (**)P<0.01, (****)P<0.0001.

FIGS. 5A-G. IFN-γ induces activation induced cell death of tumor-specific T cell loss through and impairs anti-tumor memory responses. (A) T cells were purified from TRAMP-C2 tumor bearing mice and subsequently cultured in vitro with the indicated concentrations of IFN-γ. (B) Cells were harvested at 72 hours post IFN-γ stimulation and analyzed for active Caspase-3 expression among different CD8 subsets. (C) IFN-γ receptor expression in different CD8+ T cell subsets. (D) Peripheral blood mononuclear cells (PBMC) from a nivolumab-treated patient with metastatic melanoma were stimulated in vitro with different concentrations of IFN-γ and harvested 48 hours later. Annexin V expression in CD8+ T cell subsets after stimulation with different concentration of IFN-γ. Data described as mean±SEM. (E) 8-weeks old C57BL/6j mice were challenged with TRAMP-C2 tumor on day 0 and treated with checkpoint inhibitors on day 3,6, and 9. 90 days after tumor implantation, tumor free mice from CTLA-4 blockade or combination treatment groups were subsequently rechallenged with either TRAMP-C2 or MC-38 tumor models. Aged control sibling mice without prior tumor challenge were used as control mice. (F) Mice were rechallenged with TRAMP-C2 tumors. (G) Mice were rechallenged with MC-38 tumors. FIGS. 5A-C were from 8 mice per group in two independent experiments. In order to get sufficient amount of tumor free mice in FIGS. 5E-G, each treatment group consisted of 30-45 mice per group. The numbers of tumor-free mice for rechallenge were labeled in the figures correspondingly. Statistical analyzes were calculated by one-way ANOVA with post-hoc Tukey test in FIG. 5B and two-way ANOVA with post-hoc test in FIGS. 5F and G. (*)P<0.05, (**)P<0.01, (***)P<0.001, (****)P<0.0001.

FIGS. 6A-N. Effective Anti-Tumor Response With Dual Checkpoint Blockade Is Restored When T Cells Are Made Unresponsive To IFN-γ. (A) Age and gender matched wild type (WT) or IFN-γR knockout (RKO) C57BL/6j mice were implanted with tumors at day 0 and treated with checkpoint blockade on day 3, 6, and 9. (B) Comparison of tumor growth curves in CTLA-4 blockade treatment. (C) Comparison of tumor growth curves in isotype control treatment. (D) Comparison of tumor growth curves with combination blockade treatment. (E) WT or RKO mice were treated with checkpoint blockade and harvested at day 28. (F) Total numbers of CD8+Spas-1 T cells. (G) WT mice were myeloablated (10.5 Gy) and given an adoptive transfer of bone marrow cells from CD45.2 RKO and CD45.1 congenic WT mice at 1:1 ratio. Chimera mice were subsequently implanted with TRAMP-C2 tumors and treated with checkpoint inhibitors on day 33, 36, and 39 post bone marrow transplant. (H) Mice underwent tail bleeding and were checked for chimerism 30 days post bone marrow transplantation. (I). Tumor-draining lymph nodes were harvested on day 58. Different CD8+ subsets were pre-gated on flow cytometry and investigated for chimerism. (J) Mice were challenged with TRAMP-C2 tumors and splenocytes were harvested either at Day 11 (LTB) or Day 50 (HTB). MFI expression levels of PD-1 among CD8+ T cells. (K) Tim-3 expression among CD8+ T cells (L) KLRG1 expressions among CD8+ T cells. (M) MFI expression levels of IFN-γ in CD4+ T cells. (N) MFI expression levels of IFN-γ in CD8+ T cells. FIGS. 6A-D were from 8 mice per group. FIGS. 6E-F were from separate experiments with 5 mice per group. For FIGS. 6G-I, each treatment consists of 5 chimera mice per group with total 20 chimera mice in this experiment. FIGS. 6J-N were from 5 mice per group. FIGS. 6B-D were analyzed by two-way ANOVA with post-hoc test. FIGS. 6F and I were analyzed by one-way ANOVA with post-hoc Tukey test. FIGS. 6J-N were analyzed by Student's t test. (*)P<0.05, (**)P<0.01, (***)P<0.001, (****)P<0.0001.

FIGS. 7A-G. (A) Flow gating strategy of different CD8 subsets and K562 cancer cells. (B) Annexin V staining between untransfected CD8 T cells and CAR-19 T cells after co-cultured with K-562-19 for 24 hours in vitro. (C) To investigate the effect of IFN-gamma inducing apoptosis, different concentrations of recombinant human IFN-gamma were added into cell cultures. Figures demonstrate Annexin V expression levels between untransfected CD8 T cells and CAR-19 T cells. (D) K-562 without CD-19 ligand expression were used as control. Figure demonstrated the differences between activated versus un-activated CAR-19 T cells under the influence of human recombinant IFN-gamma proteins. (E) Time points of Annexin V expression. (F) Flow cytometry analysis of IFN-gamma receptor expression among difference CD8 subsets. (G) Activation status between untransfected CD8 T cells and CAR-19 T cells.

FIGS. 8A-G. ADCC-mediated depletion of activated CD4+ and CD8+ T cells post checkpoint blockade. C57BL/6j WT mice were injected with TRAMP-C2 (1×106 cells/mouse) in the right flank on Day 0 and treated with different checkpoint inhibitors. (A) Illustration of anti-PD-1 and anti-PD-1 DANA monoclonal antibodies. (B) EL4 cells expressing PD-1 were incubated with the indicated PD-1 antibodies followed by secondary detection with anti-mouse IgG. (C) HEK293 cells overexpressing mouse PD-1 were incubated with mouse PD-L1 extracellular domain fused to human IgG1+/−the indicated PD-1 antibodies followed by secondary detection with anti-human IgG. (D) Schema of animal studies. (E) Pictures of draining lymph nodes harvested on Day 49 after different immunotherapy treatments. (F) Total CD4+ T cells from draining lymph node (G) Total CD8+ T cells from draining lymph nodes. Data of A-E were collected from 4 mice per group. Statistical differences were calculated by one-way ANOVA with post-host Tukey test. (*)P<0.05, (**)P<0.01.

FIGS. 9A-I. Expansion of antigen-specific T cells post checkpoint blockade. Mice were implanted with TRAMP-C2 on Day 0 and treated with checkpoint inhibitors on Day 40, 43, 46 and harvested on Day 49. (A) Flow gating strategy of antigen specific T cells. (B) Total numbers of CD4+ T cells in spleens. (C) Total numbers of CD8+ T cells in spleens (D) Total numbers of CD8+Spas-1 T cells in spleens. (E) Total numbers of CD8+Spas-1 T cells in draining lymph nodes. (F) Gating strategy of CD4+Foxp3+ regulatory T cells in spleens (G) Total numbers of regulatory T cells in spleens (H) Percentage of IFN-γ secretion from CD4+ T cells (I) Percentage of IFN-γ secretion from CD8+ T cells. Data were collected from 8 mice per group from two independent experiments. Statistics were analyzed by one-way ANOVA with post-host Tukey test. (*)P<0.05, (**)P<0.01, (***)P<0.001.

FIGS. 10A-C. MHC/peptide multimer staining of antigen-specific T cellis in a melanoma patient. A treatment-naïvee patient with metastatic melanoma underwent debulking surgery and then received four doses of ipilimumab plus nivolumab at three weeks interval, and followed by maintenance with nivolumab. PBMCs were collected from different time points, including baseline (without any treatment), after two cycles of ipilimumab with nivolumab treatments, and three months after the 4th cycle of ipilimumab and nivolumab. (A) Flow gating strategy is shown. (B) After pre-gating with Aqua-CD45+CD3+CD8+, cells were analyzed with control multimers. (C) After pre-gating with Aqua-CD45+CD3+CD8+, cells were analyzed by MART-1 expression.

FIGS. 11A-C. Responses induced by IFN-γ post checkpoint blockade. Splenocytes were isolated from tumor-bearing mice treated with checkpoint blockade at day 3, 6, 9 post TRAMP-C2 implantation. (A) Annexin V expression among Spas-1, Spas-2, or MHC/peptide multimer-negative CD8+ T cells. (B) MFI expression levels of Annexin V in different CD8 subsets. (C) Mice were implanted with TRAMP-C2 (106 cells per mouse) and treated with checkpoint inhibitors. Serum samples were collected two days after the last checkpoint inhibitor treatment and were analyzed for chemokine levels. Data were collected from 5 mice per group. Statistical analyses were calculated by one-way ANOVA with post-hoc Tukey test. (****)P<0.0001

FIGS. 12A-D. IFN-γ secretion from T cells induced apoptosis of antigen-specific T cells. Mice implanted with TRAMP-C2 tumors were treated with checkpoint inhibitors on day 3, 6, and 9. Spleens were harvested on Day 11 and IFN-γ expression was analyzed in CD4+ and CD8+ T cell subsets by flow cytometry after stimulation with PMA/Ionomycin and Golgi-Stop/Golgi-plug for 4 hours in vitro (A) IFN-γ expression in CD4+ T cells. (B) IFN-γ expression in CD8+ T cells. (C) Splenocytes were harvested from TRAMP-C2 bearing mice. Gating strategies of Spas-1hi and Spas-11o CD8 T cell clones. (D) Caspase-3 expression among different CD8+ Spas-1 subsets at different time points. Data were collected from 10 mice per group in two independent experiments in FIGS. 12A-B. In FIGS. 12C and D, data were collected from 5 mice per group. Statistical analyses were calculated by either one-way ANOVA (FIGS. 12A and B) or two-way ANOVA (FIGS. 12C and D) with post-hoc Tukey test. (*)P<0.05, (**)P<0.001,(****)P<0.0001

FIGS. 13A-G. Memory anti-tumor responses are compromised by combination checkpoint blockade. Mice were challenged with TRAMP-C2 tumors and treated with checkpoint inhibitors on day 3, 6, and 9. Tumor draining lymph nodes were harvested on day 28 and checked for antigen specific memory T cell subsets by flow cytometry. (A) Flow cytometry gating of CD8+ memory subsets. (B-D) Percentage of different CD8+ effector memory subsets. (E-G) Percentage of different CD8+ central memory subsets. Data were generated with 5 mice per group. Statistical analyzes were calculated by Student's t test. (*)P<0.05

FIGS. 14A-E. T cell exhaustion in low and high tumor burden. Mice were challenged with TRAMP-C2 tumors (106 per mouse) and splenocytes were harvested either at Day 11 (low tumor burden-LTB) or Day 50 (high tumor burden-HTB). (A) Schema of animal study. (B and C) Flow gating strategy and T cell exhaustion status. (D) PD-1 expression among CD4 T cells (E) Tim-3 expression among CD4 T cells. Data were generated with 4-5 mice per group. Statistical analyzes were calculated by Student's t test. (*)P<0.05, (**)P<0.01

FIG. 15. Goldilocks phenomena of checkpoint inhibitor blockade. Our results indicate while insufficient immune stimuli fail to protect against tumor growth (PD-1 blockade in our experiment), exceeding an immune stimulatory threshold may trigger an immunomodulatory regulation mechanism that suppresses anti-tumor responses via IFN-γ. Achieving the optimal magnitude of immune stimulation may be critical for successful immunotherapy strategies and achieving optimal tumor control as well as long-term outcome for patients.

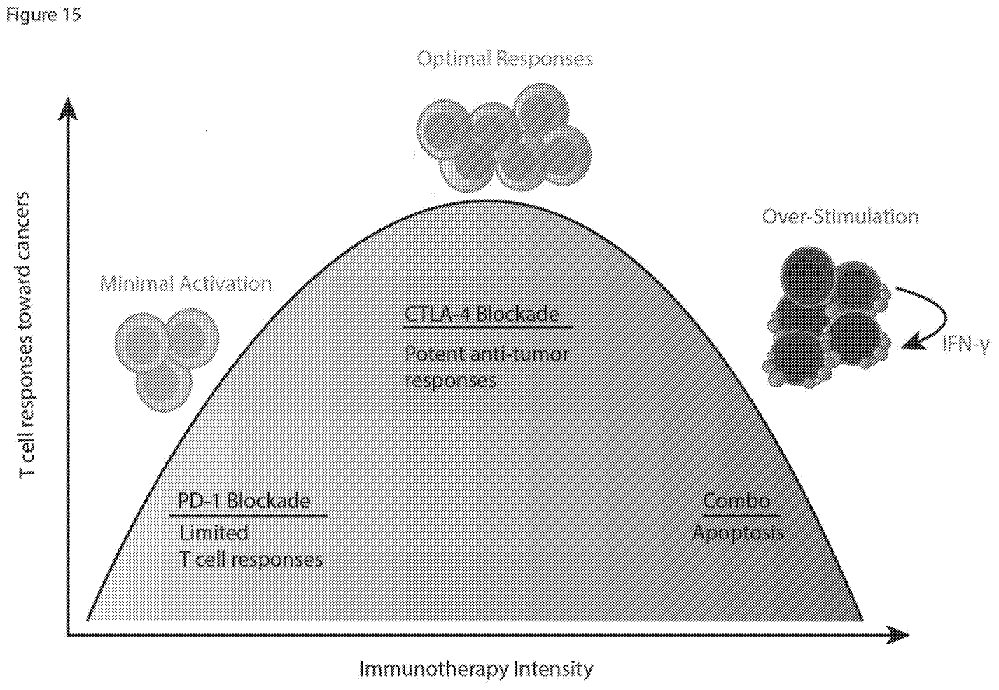

FIG. 16. The difference in T cell exhaustion status between low and high tumor burdens can contribute to different outcomes of checkpoint inhibitor immunotherapies. In low tumor burdens, the combination of checkpoint inhibitors can induce high levels of IFN-γ secretion, which in turns results in activation induced cell death (AICD) of antigen specific T cells via expression of caspase genes. In contrast, combination checkpoint inhibitors in high tumor burdens can partially reinvigorate exhausted T cells, resulting in moderate amounts of IFN-γ secretion, which can enhance recognition of cancer cells by T cells through increasing MHC I expression.

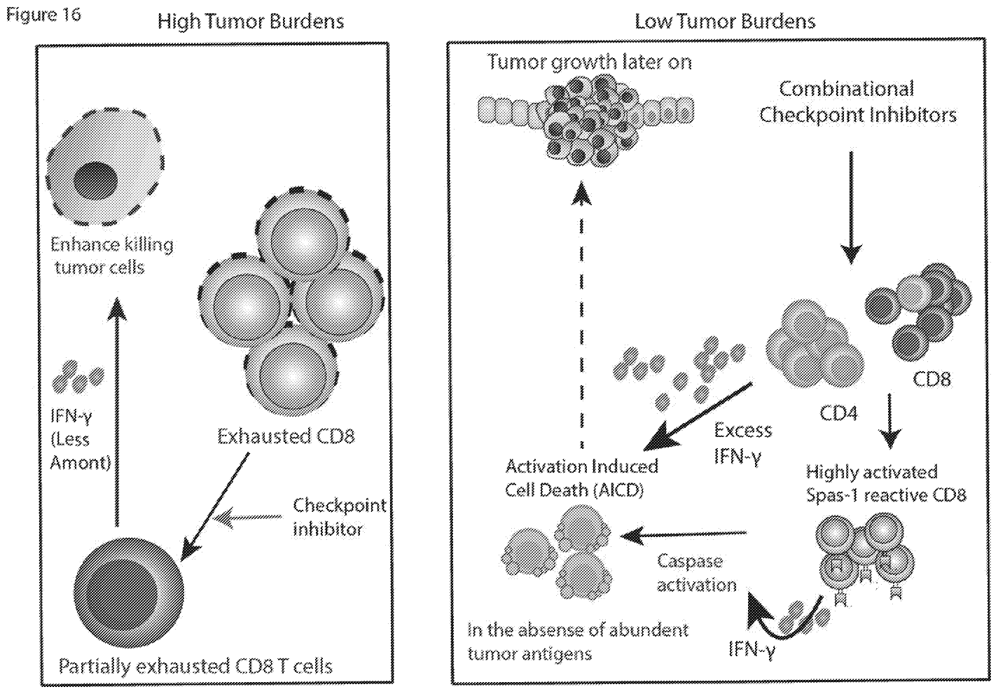

### DEFINITIONS

A polynucleotide or polypeptide sequence is “heterologous” to a cell if it originates from a different cell, or, if from the same cell, is modified from its original form. For example, when a T-cell receptor is said to be heterologous to a T-cell, it means that the receptor or coding sequence thereof is from a first T-cell whereas the receptor is present or expressed in a second T-cell or the T-cell receptor is modified from its original form. For example the heterologous receptor can be expressed in the second T-cell by cloning or otherwise obtaining the coding sequence of the receptor from the first T-cell and inserting the coding sequence into the second T-cell such that the receptor is expressed in the second T-cell.

“Tumor-specific” means that the T-cell targets to tumor (cancer) cells. For example a tumor-specific T-cell can have a receptor that has binding affinity for a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell (e.g., under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The term “interfering RNA” or “RNAi” or “interfering RNA sequence” refers to double-stranded RNA (i.e., duplex RNA) that targets (i.e., silences, reduces, or inhibits) expression of a target gene (i.e., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA typically has substantial (e.g., at least 70%, 80%, 90%, or 95%) or complete identity to the target gene. The sequence of the interfering RNA can correspond to the full length target gene, or a subsequence thereof. Interfering RNA includes small-interfering RNA″ or “siRNA,” i.e., interfering RNA of about 15-60, 15-50, 15-50, or 15-40 (duplex) nucleotides in length, more typically about, 15-30, 15-25 or 19-25 (duplex) nucleotides in length, e.g., about 20-24 or about 21-22 or 21-23 (duplex) nucleotides in length (e.g., each complementary sequence of the double stranded siRNA is 15-60, 15-50, 15-50, 15-40, 15-30, 15-25 or 19-25 nucleotides in length, e.g. about 20-24 or about 21-22 or 21-23 nucleotides in length, and the double stranded siRNA is about 15-60, 15-50, 15-50, 15-40, 15-30, 15-25 or 19-25 e.g., about 20-24 or about 21-22 or 21-23 base pairs in length). siRNA duplexes may comprise 3′ overhangs of about 1 to about 4 nucleotides, e.g., of about 2 to about 3 nucleotides and 5′ phosphate termini. The siRNA can be chemically synthesized or may be encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., PNAS USA 99: 9942-7 (2002); Calegari et al., PNAS USA 99: 14236 (2002); Byrom et al., Ambion TechNotes 10(1): 4-6 (2003); Kawasaki et al., Nucleic Acids Res. 31: 981-7 (2003); Knight and Bass, Science 293: 2269-71 (2001); and Robertson et al., J. Biol. Chem. 243: 82 (1968)). In some embodiments, dsRNA are at least 50 nucleotides to about 100, 200, 300, 400 or 500 nucleotides in length. A dsRNA may be as long as 1000, 1500, 2000, 5000 nucleotides in length, or longer. The dsRNA can encode for an entire gene transcript or a partial gene transcript.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA.

The term “antisense nucleic acid” as used herein means a nucleotide sequence that is complementary to its target e.g. a IFN-γ receptor transcription product. The nucleic acid can comprise DNA, RNA or a chemical analog, that binds to the messenger RNA produced by the target gene. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, “treating” and “treatment includes: (i) inhibiting the disease or condition, i.e., arresting its development; (ii) relieving the disease or condition, i.e., causing regression of the disease or condition; or (iii) relieving the symptoms resulting from the disease or condition, i.e., relieving pain without addressing the underlying disease or condition.

### DETAILED DESCRIPTION OF THE INVENTION

### Introduction

It has been discovered that treatment of cancer patients with checkpoint inhibitors can result in deletion of tumor-specific T-cells and that this effect is mediated by interferon-gamma (IFNγ). It has been further discovered that the deleterious effect of IFNγ on T-cells can be countered by knocking out the IFNγ receptor in the T-cells, rendering the T-cells immune from the negative effects of IFNγ and thus remaining available to target cancer cells.

Accordingly, in some aspects, tumor-specific T-cells are provided that do not express a functional IFNγ receptor. Such cells can be administered to treat cancer in a human. In some embodiments, the T-cells can be T-cells obtained from the human and then altered to block expression of a functional IFNγ receptor. In some embodiments, the T-cells can be further modified, for example to express a heterologous protein that targets the T-cells to cancer cells. Such heterologous proteins can include, for example chimeric antigen receptors or T-cell receptors that are heterologous to the T-cells. The T-cells can be administered with, or as a complementary treatment with, checkpoint inhibitors. Exemplary check point inhibitors include but are not limited to CTLA-4 inhibitors, PD-1 inhibitors or combinations thereof.

As JAK functions downstream of the IFNγ receptor, the effect of JAK inhibitors was also determined in the context of checkpoint inhibitors. It was discovered that combination of a JAK inhibitor with anti-CTLA-4 and anti-PD-1 checkpoint inhibitors were effective in reducing incidence of tumors in a mouse cancer model. Accordingly, an alternative to (or possible combination with) knock out of a functional IFNγ receptor in T-cells is to administer to a human having cancer an effective amount of a JAK inhibitor in combination with a CTLA-4 inhibitor and a PD-1 inhibitor.

### T-Cells

Any type of T-cells can be modified to block expression of a functional IFNγ receptor. In some embodiments, the T-cell will be a CD8⁺ T-cell or a CD4+ T cell. These can include T-cells that have been transduced with chimeric antigen receptors (CAR) or T cell receptors (TCR) that target the relevant antigen in tumors both on the tumor cell or within the tumor microenvironment.

T cells can be obtained from a number of sources, including but not limited to peripheral blood mononuclear cells, bone marrow, lymph node tissue, cord blood, thymus tissue, tissue from a site of infection, ascites, pleural effusion, spleen tissue, and tumors. For example, in some embodiments, cells from the circulating blood of an individual are obtained by apheresis, optionally followed by enrichment for T-cells, for example by affinity (e.g., antibody)-based cell sorting. In some embodiments, the T-cells can be cryopreserved and/or expanded before use. Exemplary details of T-cell manipulation can be found in, e.g., U.S. Pat. No. 9,394,368.

In some embodiments, the T-cells are obtained from the individual, modified as described herein, and returned to the same individual. Alternatively, T-cells can be obtained from one individual and administered to a different individual. In some embodiments, major HLA loci will be matched between the donor individual and the recipient to avoid rejection of the T-cells by the recipient.

### Knocking Out Receptor

T-cells do that not express a functional Interferon-γ (IFN-γ) receptor can be generated in a number of ways. In some embodiments, point mutations or deletions can be induced in the coding sequence of the IFN-γ receptor or in promoter or other transcriptional or translational regulator regions in DNA to result in a non-functional IFN-γ receptor. Thus in some embodiments the DNA of the T-cell can include a coding sequence of a functional IFN-γ receptor that is not expressed. Alternatively, the DNA of the T-cell can encode a non-functional IFN-γ receptor that includes one or more amino acid change, deletion or addition that impairs or eliminates IFN-γ receptor function (e.g., the ability to bind IFN-γ or to trigger downstream signaling based on IFN-γ binding). In yet other embodiments, all or part of the IFN-γ receptor gene can be deleted, thereby preventing expression of a functional IFN-γ receptor.

In yet other embodiments, expression of a native T-cell IFN-γ receptor can be inhibited by introduction of one or more agent that inhibits expression of the IFN-γ receptor. Exemplary agents include, but are not limited to, RNAi, siRNA, or antisense polynucleotides that are complementary or substantially (e.g., at least 75%, 80%, 85%, 90%, or 95%) complementary to all or a subsequence of at least 15, 20, 25, 30, 50, or more nucleotides of native RNA encoding the IFN-γ receptor.

Any method of genetic manipulation can be used to introduce the above-described mutations to block IFN-γ receptor expression. In some embodiments, a double-strand break (DSB) or nick for can be created by a site-specific nuclease in or near the target gene (e.g., the IFN-γ receptor gene). Exemplary targeted nucleases include but are not limited to zinc-finger nuclease (ZFN) or TAL effector domain nuclease (TALEN), or the CRISPR/Cas9 system with an engineered crRNA/tract RNA (single guide RNA) to guide specific cleavage. See, for example, Burgess (2013) Nature Reviews Genetics 14:80-81, Urnov et al. (2010) Nature 435(7042):646-51; United States Patent Publications 20030232410; 20050208489; 20050026157; 20050064474; 20060188987; 20090263900; 20090117617; 20100047805; 20110207221; 20110301073 20110301073;20130177983; 20130177960 and International Publication WO 2007/014275, WO2003087341; WO2000041566; WO2003080809. Nucleases specific for targeted genes can be utilized such that a transgene construct is inserted by either homology directed repair (HDR) or by end capture during non-homologous end joining (NHEJ) driven processes.

### Tumor-Specific Proteins Expressed in T-Cells

In some embodiments, the T-cells are tumor-specific. Tumor-specific T-cells include one or more receptor that targets the T-cell to a cancer cell. In some embodiments, the T-cell has a naturally-occurring T-cell receptor that targets the T-cell to a cancer epitope. In other embodiments, the T-cell can be modified by introduction of a heterologous T-cell receptor specific for a cancer epitope. In yet other embodiments, the T-cell can be modified to express a chimeric antigen receptor that targets a cancer epitope. In other embodiments, the T-cell can be bound to a bispecific binding agent where the binding agent binds to the T-cell (e.g., at a surface protein on the T-cell) and separately has a binding affinity for a cancer epitope.

T-cells having a naturally-occurring T-cell receptor that targets the T-cell to a cancer epitope can be isolated from T-cell populations. In some embodiments, the T-cells are from an individual having a cancer that expresses the cancer epitope. T-cells that target the cancer epitope can be enriched using affinity selection in vitro using the cancer epitope or active fragment thereof.

Alternatively, T-cells can be modified by introduction of a heterologous T-cell receptor specific for a cancer epitope. Methods of doing so are described, in, e.g., Rapoport et al., Nature Medicine 21:914-921 (2015).

In some embodiments, the cancer epitope targeted by the tumor-specific T-cell is CD19, CD20, CD22, ROR1, mesothelin, CD33/IL3Ra, c-Met, PSMA, Glycolipid F77, EGFRvIII, GD-2, NY-ESO-1 TCR, MAGE A3 TCR. In some embodiments, the cancer epitope is one of: Differentiation antigens such as MART-1/MelanA (MART-1), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.291\BCAA, CA 195, CA 242, CA-50, CAM43, CD68\Pl, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In some embodiments, the T-cell is further modified to lack a naturally-occurring T-cell receptor (this can also occur when the T-cell is modified to express a chimeric antigen receptor). See, e.g., U.S. Pat. No. 9,181,527 describing methods of knocking out endogenous T-cell receptors.

In yet other embodiments, the T-cell can be modified to express a chimeric antigen receptor (CAR) that targets a cancer epitope. Exemplary CAR receptors are reviewed in Jackson et al., Nat Rev Clin Oncol. 2016 Jun;13(6):370-83. In some embodiments, the CAR comprises an antibody or antibody fragment that includes a cancer epitope (e.g., as listed above)-binding domain (e.g., a humanized antibody or antibody fragment that specifically binds to the cancer epitope), a transmembrane domain, and an intracellular signaling domain (e.g., an intracellular signaling domain comprising a costimulatory domain and/or a primary signaling domain). A number of CAR designs have been described including but not limited to those described in U.S. Pat. Nos. 9,522,955; 9,511,092; 9,499,629; 9,499,589; 9,447,194; and 9,394,368. In some embodiments, the binding domain of CAR comprises a scFv, comprising the light (VL) and heavy (VH) variable fragments of a cancer epitope-specific monoclonal antibody joined by a flexible linker. The intracellular signaling portions of the CAR can differ. In some embodiments (e.g., first generation CARs), the CAR only has the signal transduction domain of the CD3-zeta chain (CD3ζ) or Fc receptor γ (FcRγ). In other embodiments, the CAR further comprises one or more co-stimulatory domains (including but not limited to one or more of CD28, 4-1BB, or OX40), which in some embodiments lead to the enhanced cytotoxicity and cytokine secretion along with prolonged T cell persistence.

In some embodiments, viral or non-viral based gene transfer methods can be used to introduce nucleic acids encoding a heterologous T-cell receptor or a CAR T-cell receptor into T-cells as desired. Non-viral vector delivery systems include DNA plasmids, naked nucleic acid, and nucleic acid complexed with a delivery vehicle such as a liposome, lipid nanoparticle or poloxamer Viral vector delivery systems include DNA and RNA viruses, which have either episomal or integrated genomes after delivery to the cell. Methods of non-viral delivery of nucleic acids include electroporation, lipofection, microinjection, biolistics, virosomes, liposomes, lipid nanoparticles, immunoliposomes, polycation or lipid:nucleic acid conjugates, naked DNA, mRNA, artificial virions, and agent-enhanced uptake of DNA. In some embodiment, one or more nucleic acids are delivered as mRNA. In some embodiments, capped mRNAs are used to increase translational efficiency and/or mRNA stability. See, e.g., U.S. Pat. Nos. 7,074,596 and 8,153,773.

In other embodiments, the T-cell can be bound to a bispecific binding agent where the binding agent binds to the T-cell (e.g., at a surface protein on the T-cell, for example including but not limited to CD3) and separately has a binding affinity for a cancer epitope (for example, but not limited to CD20). Bi-specific antibodies capable of targeting T cells to tumor cells have been identified and tested for their efficacy in the treatment of cancers. Blinatumomab is an example of a bispecific anti-CD3-CD19 antibody in a format called BiTE™ (Bi-specific T-cell Engager) that has been identified for the treatment of B-cell diseases such as relapsed B-cell non-Hodgkin lymphoma and chronic lymphocytic leukemia (Baeuerle et al (2009)12:4941-4944). The BiTE™ format is a bi-specific single chain antibody construct that links variable domains derived from two different antibodies. Additional bi-specific antibodies include but are not limited to those described in WO 2015006749 and WO 2016110576. Such bi-specific binding agents can be mixed with a T-cell that does not express a functional IFN-γ receptor, thereby rendering the T-cell tumor-specific by way of the affinity of the bi-specific agent for a cancer epitope.

Treatment and Combination with Checkpoint Inhibitors

The tumor-specific T-cells lacking expression of a functional IFN-γ receptor can be used to treat cancer in a human. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the T-cells described herein include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases). In one embodiment, the epitope-binding portion of the CAR is designed to treat a particular cancer. In some embodiments, a CAR or T-cell receptor targeting CD19 can be used to treat cancers and disorders including but are not limited to pre-B ALL (pediatric indication), adult ALL, mantle cell lymphoma, diffuse large B-cell lymphoma, salvage post allogenic bone marrow transplantation, and the like. In another embodiment, a CAR or T-cell receptor targeting CD22 to treat diffuse large B-cell lymphoma. In one embodiment, cancers and disorders include but are not limited to pre-B ALL (pediatric indication), adult ALL, mantle cell lymphoma, diffuse large B-cell lymphoma, salvage post allogenic bone marrow transplantation, and the like can be treated using a combination of CAR or T-cell receptors targeting CD19, CD20, CD22, and ROR1. In one embodiment, a CAR or T-cell receptor targeting mesothelin can be used to treat mesothelioma, pancreatic cancer, and ovarian cancer. In one embodiment, a CAR or T-cell receptor targeting CD33/IL3Ra can be used to treat acute myelogenous leukemia. In one embodiment, a CAR or T-cell receptor targeting c-Met can be used to treat triple negative breast cancer, and non-small cell lung cancer. In one embodiment, a CAR or T-cell receptor targeting PSMA can be used to treat prostate cancer. In one embodiment, a CAR or T-cell receptor targeting Glycolipid F77 can be used to treat prostate cancer. In one embodiment, a CAR or T-cell receptor targeting EGFRvIII can be used to treat glioblastoma. In one embodiment, a CAR or T-cell receptor targeting GD-2 can be used to treat neuroblastoma, and melanoma. In one embodiment, a CAR or T-cell receptor targeting NY-ESO-1 TCR can be used to treat myeloma, sarcoma, and melanoma. In one embodiment, a CAR or T-cell receptor targeting MAGE A3 TCR can be used to treat myeloma, sarcoma, and melanoma.

The T cells described herein may be administered either alone, or as a pharmaceutical composition in combination with diluents and/or with other components such as IL-2 or other cytokines or cell populations. Briefly, pharmaceutical compositions may comprise a T-cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention can be formulated for intravenous administration.

Pharmaceutical compositions as described herein may be administered in a manner appropriate to the disease to be treated. The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions as described herein to be administered can be determined by a physician with consideration of individual differences in age, weight, tumor size, extent of infection or metastasis, and condition of the patient (subject). In some embodiments, a pharmaceutical composition comprising the T cells described herein may be administered at a dosage of 10⁴ to 10⁹ cells/kg body weight, e.g., 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions are administered to a patient by intradermal or subcutaneous injection. In another embodiment, the T cell compositions are administered by i.v. injection. The compositions of T cells may be injected directly into a tumor, lymph node, or site of infection.

In certain embodiments, the T-cells as described herein are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p′7056 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cell compositions as described herein are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the T-cells as described herein. In an additional embodiment, expanded cells are administered before or following surgery.

As noted herein, inhibiting immune checkpoint pathways, while useful in treating various cancers, can result in an increase in IFN-γ. Thus, in some embodiments in T-cells described herein are used in combination with one or more immune pathway checkpoint inhibitor. In some embodiments the immune pathway checkpoint inhibitor is selected from a PD-1 inhibitor, a PD-L1 inhibitor, a CTLA-4 inhibitor, a Lag-3 inhibitor, a TIM-3 inhibitor, or a combination thereof. Exemplary inhibitors can include but are not limited to antibodies that bind to the immune pathway checkpoint protein in question (e.g., PD-1 or CTLA-4). PD-1 and CTLA-4 inhibition is discussed in, e.g., Buchbinder, and Desai, Am J Clin Oncol. 2016 February; 39(1): 98-106. Exemplary CTLA-4 antibodies include but are not limited to Ipilimumab (trade name Yervoy™) as well as those described in, e.g., WO 2001/014424, U.S. Pat. No. US 7,452,535; 5,811,097. Exemplary PD-1 antibodies include but are not limited to Pembrolizumab (formerly MK-3475 and lambrolizumab, trade name Keytruda™) as well as those described in, e.g., U.S. Pat.t No. 8,008,449 and Zarganes-Tzitzikas, et al., Journal Expert Opinion on Therapeutic Patents Volume 26, 2016, Issue 9. Inhibitors of the immune pathway checkpoints can be administered before, after or simultaneously with administration of the T-cells as described herein. The dosage of the above treatments to be administered to a patient will vary with the precise nature of the condition being treated and the recipient of the treatment. The scaling of dosages for human administration can be performed according to art-accepted practices.

In alternate embodiments, instead of providing T-cells that do not express a functional IFN-γ receptor, an antibody or other binding agent that binds to IFN-γ can be administered to the human. This will inactivate or eliminate IFN-γ sufficiently to interfere with IFN-γ′s negative effect on T-cells in the human. Thus in some embodiments, an antibody or other binding agent that binds to IFN-γ is administered to the human in conjunction with (e.g., simultaneously with or before or after within 1-30 days) one or more immune checkpoint inhibitors as described herein (including but not limited to, for example anti-PD-1 and anti-CTLA4 agents). In some embodiments, a T-cell is also administered to the human as described herein albeit the T-cells can be, but need not be, blocked for IFN-γ include a receptor (heterologous or native, and optionally CAR) that targets a cancer epitope as described herein.

### JAK Inhibitors

As noted herein, in some embodiments, an effective amount of one or more Janus kinase (JAK) inhibitor is administered to a human having cancer, optionally in combination with an anti-PD-1 agent and an anti-CTLA4 agent. Agents administered in combination can be administered simultaneously or in a mixture together, or can be administered in series. For administration in series (not simultaneous) agents should be administered within a time frame such that an initially administered agent still has the desired effect (e.g., has not been significantly degraded or excreted from the body) by the time later agents in the combination are administered to have the desired effect, e.g., cancer cell killing or reduction of tumor incidence.

Exemplary JAK inhibitors include but are not limited to Ruxolitinib (trade names Jakafi/Jakavi), Tofacitinib (trade names Xeljanz/Jakvinus, formerly known as tasocitinib and CP-690550, Oclacitinib (trade name Apoquel), Baricitinib (trade name Olumiant), Filgotinib (G-146034, GLPG-0634), Gandotinib (LY-2784544), Lestaurtinib (CEP-701), Momelotinib (GS-0387, CYT-387), Pacritinib (SB1518), PF-04965842, Upadacitinib (ABT-494), Peficitinib (ASP015K, JNJ-54781532), or Fedratinib (SAR302503).

### EXAMPLES

The following examples are offered to illustrate, but not to limit the claimed invention.

### Example 1

Combination checkpoint inhibition with high tumor burden

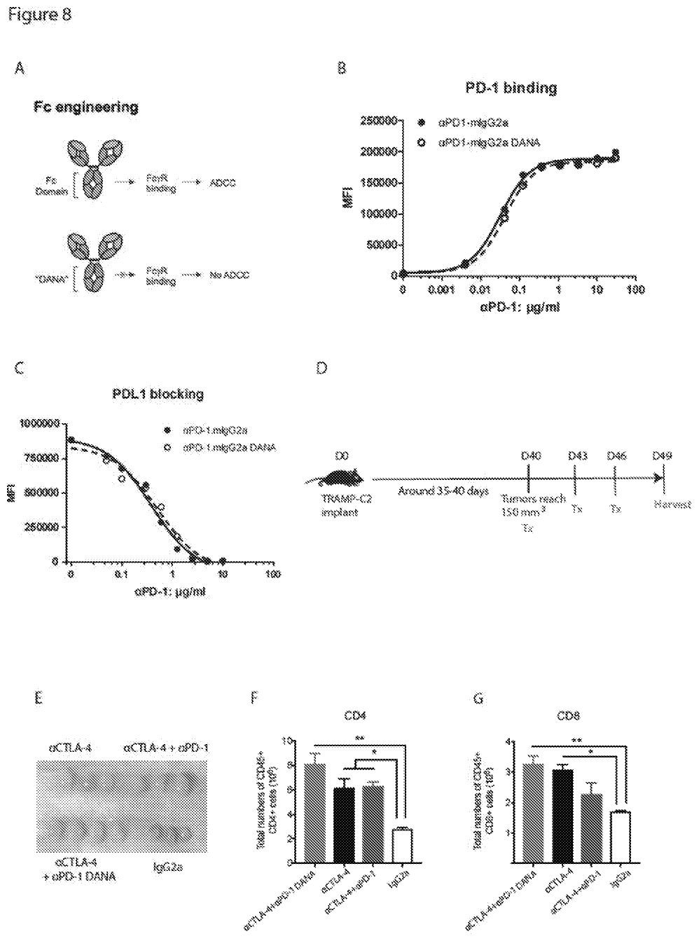

Ipilimumab is a humanized IgG1 antibody targeting CTLA-4, and one of its immunomodulatory mechanisms is engagement with FcγRIIIA to potentially antagonize or deplete regulatory T cells (E. Romano et al., Proceedings of the National Academy of Sciences of the United States of America 112, 6140-6145 (2015); M. J. Selby et al., Cancer Immunol Res 1, 32-42 (2013); T. R. Simpson et al., The Journal of experimental medicine 210, 1695-1710 (2013)). In contrast, anti-PD-1 antibodies such as nivolumab have been engineered to avoid FcγR binding to prevent depletion of activated T cells through antibody dependent cellular cytotoxicity (ADCC) (C. Wang et al., Cancer Immunol Res 2, 846-856 (2014); R. Dahan et al., Cancer cell 28, 285-295 (2015)). To mimic the anti-PD-1 antibodies used clinically, we generated an anti-PD-1 antibody without ADCC (anti-PD-1 DANA) for use in our preclinical experiments (FIGS. 8A-C). We also observed that IgG2a PD-1 blockade with intact ADCC depleted activated CD4+ and CD8+ T cells in tumor bearing mice compared to PD-1 blockade without ADCC (FIGS. 8D-G).

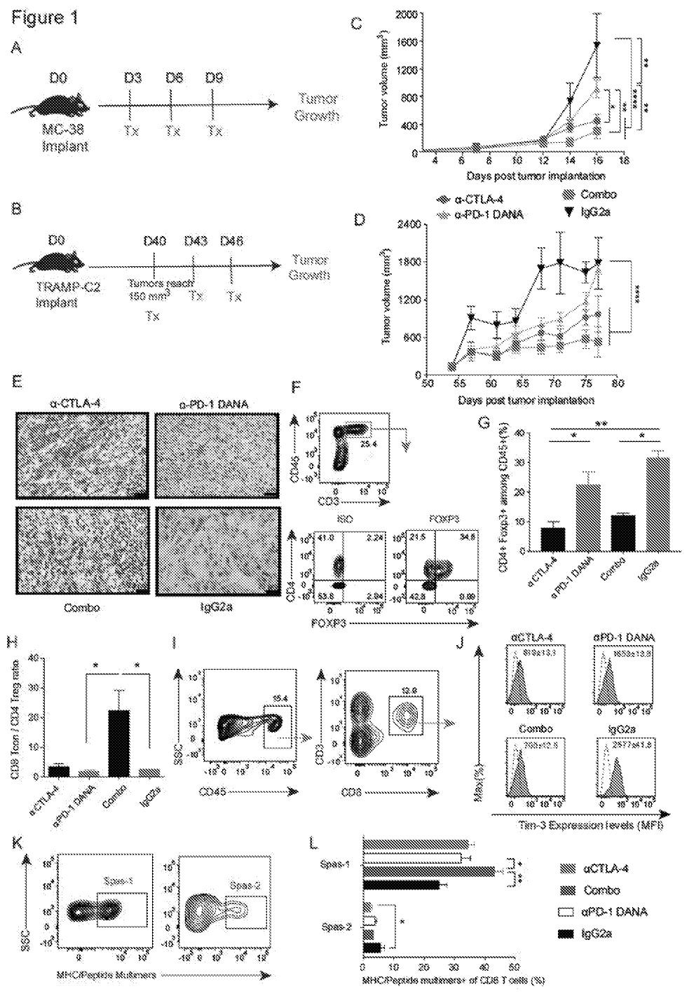

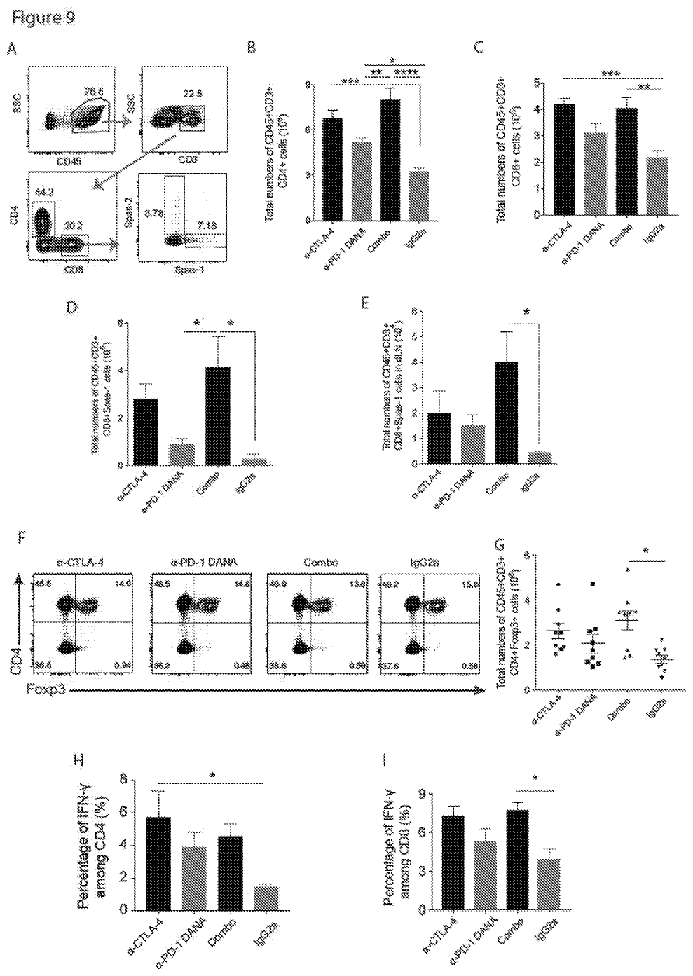

We first investigated the anti-tumor activity of single agent and combined immune checkpoint blockade in the setting of established tumors. Mice were inoculated with either MC-38 (FIG. 1A) or TRAMP-C2 cell lines on day 0 (FIG. 1B) and treated with three doses of anti-CTLA-4 alone, anti-PD-1 DANA alone, anti-CTLA4 plus anti-PD-1 DANA (combo), or IgG2a isotype control. In both models, treatment was started when tumors were palpable: on day 3, 6 and 9 for MC-38, and on day 40, 43 and 46 for TRAMP-C2. In both models, anti-CTLA-4 monotherapy demonstrated potent anti-tumor activity compared to anti-PD-1 DANA or isotype (P<0.01 and P<0.0001 for MC-38; P<0.0001 and P<0.0001 for TRAMP-C2). Combination treatment marginally improved tumor control compared to anti-CTLA-4 alone, but this was not statistically significant (FIGS. 1C-D). To further evaluate the treatment-induced effects, mice were assessed for modulated immune responses three days after the last dose of treatment. Both CD4+ and CD8+ T cells were expanded in the spleen of the combination group and anti-CTLA-4 treatment group compared to isotype (FIGS. 9A-C). We had previously described the immunodominant CD8 T cell epitope (Spas-1) that can mediate tumor rejection in the TRAMP-C2 model (M. Fasso et al., Proceedings of the National Academy of Sciences of the United States of America 105, 3509-3514 (2008)). Using MHC/peptide multimers, we tracked the changes in the frequency and number of antigen specific T cells post treatments. Consistent with the anti-tumor activity observed, combined checkpoint blockade induced the highest numbers of Spas-1 specific CD8+ T cells in draining lymph nodes compared to anti-CTLA-4, anti-PD-1 DANA, or isotype control (FIGS. 9D-E; 4.01±1.68 versus 1.99±1.18 versus 1.49±0.54 versus 0.46±0.06 respectively). Overall, these results showed that immune checkpoint blockade in mice with established tumors improved anti-tumor activity, at least in part due to the generation of a greater number of activated antigen specific T cells.

To investigate immune infiltration within the tumor microenvironment after treatment, we first evaluated pathologic changes of tumor samples harvested three days after the last treatment. In H&E staining, there were peri-tumor lymphocytic aggregates with prominent perivascular localization and intra-tumor lymphocytic penetration in combo and monotherapy treated group (FIG. 1E). Assessing the regulatory T cells within the tumors by flow cytometry, we found the frequency of CD4+Foxp3+ Treg cells in both anti-CTLA-4 and combo groups to be lower compared to isotype (FIGS. 1F-G; 7.94±1.73% versus 12.13±0.58% versus 31.55±1.80%; P<0.01 and P<0.05). Furthermore, the ratio of conventional CD8+ T cells (CD8 Tcon) to CD4+ Treg cells were significantly increased in the combo group compared to isotype (FIG. 1H; 22.40±6.78 versus 2.64±0.49; P<0.05). However, there was no significant difference between the combo group compared to anti-CTLA-4 alone.

To evaluate the exhaustion status of the infiltrating CD8+ T cells, we gated on CD8+ subsets and assessed Tim-3 expression (FIGS. 1I-J). Compared to checkpoint blockade groups, the isotype treatment group exhibited higher levels of Tim-3 expression, indicating that these CD8+ T cells are phenotypically terminal exhausted (E. J. Wherry, Nature immunology 12, 492-499 (2011)). To define the specificity of tumor-infiltrating CD8+ T cells, we gated on CD8+ T cells that recognize either dominant Spas-1 epitope or a subdominant epitope (Spas-2) (FIG. 1K), the latter of which can induce IFN-γ secretion from T cells but cannot mediate tumor rejection. Higher frequencies of dominant epitope CD8 clones were present in the combination treatment group compared to anti-PD-1 DANA or isotype treated groups (FIG. 1L; 43.08±3.57 versus 32.23±3.72 versus 24.93±2.30; P<0.05 and P<0.01 respectively). Conversely, the frequency of minor epitope CD8+Spas-2-reactive T cells were higher in the isotype group compared to anti-CTLA-4 treatment (P<0.05). Taken together, anti-CTLA-4 demonstrated potent tumor control in established tumor models by decreasing Treg cells and increasing CD8+ T cells within the tumors. The addition of anti-PD-1 DANA to anti-CTLA-4 also increased CD8+ T cells, particularly CD8+ T cells within the tumor microenvironment reactive to the immunodominant epitope Spas-1.

### Anti-PD-1 Compromises the Anti-Tumor Effects of Anti-CTLA-4 in Low Tumor Burden

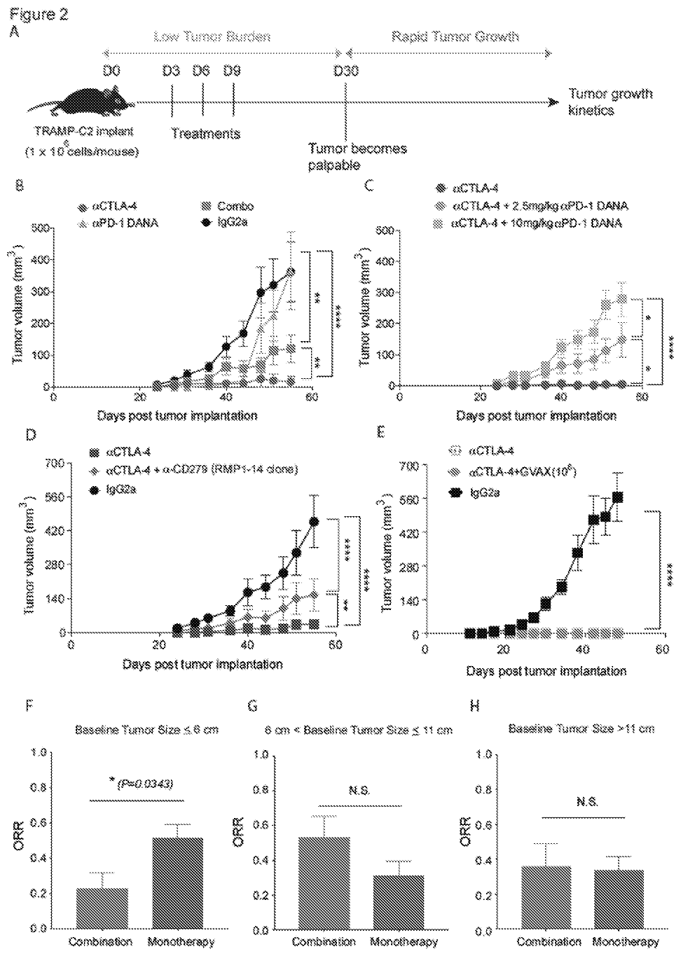

Next, we evaluated whether the combo could enhance anti-tumor responses in the setting of low tumor burdens. TRAMP-C2 has a relatively slow tumor growth rate and tumors do not become palpable until approximately 30 days post implantation. For these experiments, mice were treated on day 3, 6 and 9 (FIG. 2A). While anti-CTLA-4 strongly inhibited tumor growth compared to isotype (FIG. 2B; P<0.0001), anti-PD-1 DANA alone delayed tumor growth more transiently. However, surprisingly, the addition of PD-1 blockade attenuated the anti-tumor effects of anti-CTLA-4: mice receiving the combo had significantly larger tumors compared to mice treated with anti-CTLA-4 alone (FIG. 2B; P<0.01). To further investigate the negative contribution of anti-PD-1 DANA antibody in combination treatment, the dose of anti-PD-1 DANA antibody was titrated. When combined with anti-CTLA-4, higher dose (10 mg/kg) of PD-1 blockade treatment led to more significant dampening of anti-tumor efficacy compared to a lower dose of anti-PD-1 DANA antibody treatment (2.5 mg/kg) (FIG. 2C; P<0.05). Similar results were seen when we repeated the experiment with another clone of anti-PD-1 antibody (RMP1-14 clone) (FIG. 2D; P<0.05). In order to evaluate whether other immunotherapies could also compromise the anti-tumor effects from anti-CTLA-4 treatment, we treated mice with anti-CTLA-4 antibody combined to GM-CSF secreting cell-based cancer vaccine (GVAX). The combination of GVAX and anti-CTLA-4 showed similar anti-tumor control compared to anti-CTLA-4 alone (FIG. 2E). In summary, the addition of anti-PD-1 DANA antibody reduced the anti-tumor efficacy of CTLA-4 blockade in the low tumor burden setting.

To determine whether similar outcomes can also be observed in patients treated with dual checkpoint blockade, we analyzed 152 melanoma patients and stratified patient cohorts based on tumor burden. Patients treated with anti-PD-1 monotherapy (n=101), or the combination of anti-CTLA-4 and anti-PD-1 (n=51) were assessed for best objective response rate as determined by RECIST 1.1. Baseline tumor size was calculated per RECIST 1.1, and the baseline tumor groups were stratified into size ≤6 cm, >6 to ≤11 cm, or >11 cm. Patients with a complete or partial response were categorized as responders, and those with stable disease or progressive disease as their best response were categorized as non-responders. The responder fraction was calculated by dividing responders/all patients. Consistent with our findings in mice, patients treated with dual checkpoint blockade demonstrated significantly worse response rates compared to those treated with monotherapy in the low, but not medium or high, tumor burden settings (FIGS. 2 F-H; P<0.05). Overall, our pre-clinical and clinical data indicate that combination checkpoint blockade may paradoxically result in worse anti-tumor responses compared to monotherapy in the low tumor burden state.

Loss of Antigen-Specific T Cells with Combination Checkpoint Blockade with Low Tumor Burden

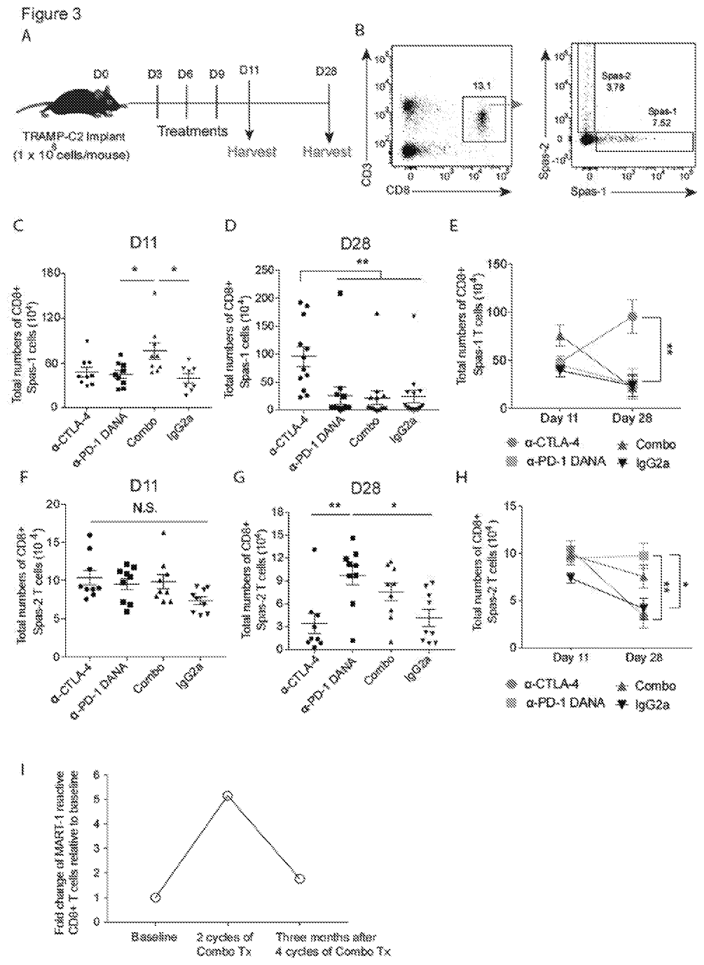

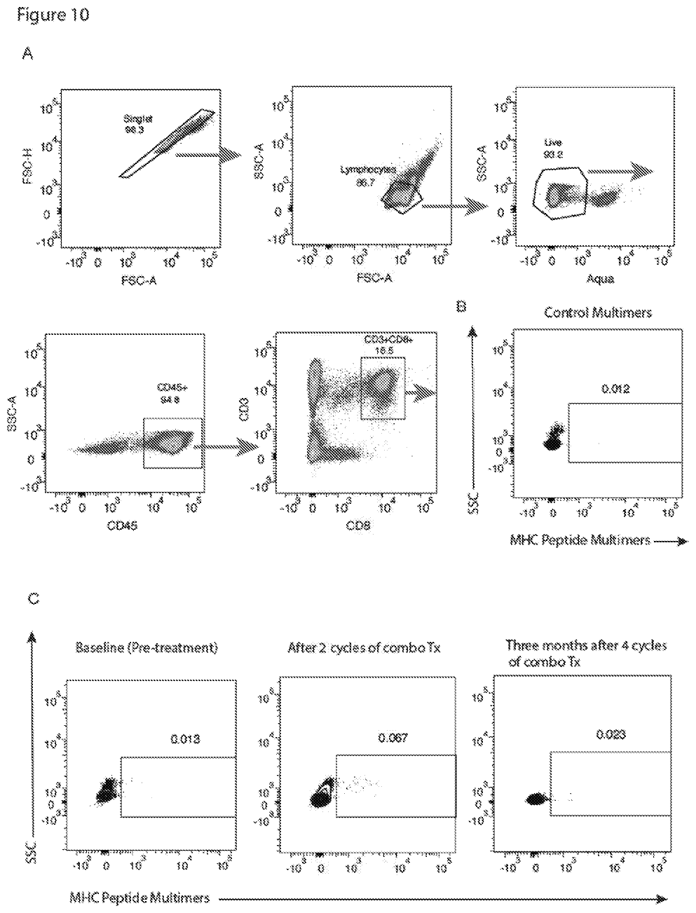

To examine the mechanism underlying this paradoxical effect, we investigated changes in the number of antigen-specific T cells at different time points in mice (FIG. 3A). Two days after the last dose of treatment, Spas-1 specific CD8+ T cells were expanded in the combination treatment group compared to either anti-PD-1 DANA or isotype (FIGS. 3B-C; 76.0±16.8 versus 45.1±7.9 versus 39.4±8.6; P<0.05). However, 28 days after the last dose of treatment, Spas-1 CD8+ T cells were found at significant levels only in the mice treated with anti-CTLA-4 (FIGS. 3D-E; P<0.01). In contrast, the Spas-2 specific CD8+ did not show significant differences on day 11 (FIG. 3F). On day 28, there was increase in total numbers of Spas-2 CD8+ T cells in anti-PD-1 DANA group compared to anti-CTLA-4 or isotype (FIGS. 3G-H). These results indicate that early treatment with anti-CTLA-4 alone allows for sustained expansion of CD8+ T cells specific for the dominant Spas-1 antigen. In contrast, anti-PD-1 DANA supports expansion of Spas-2 specific T cells. Combination treatment led to transient induction and then loss of Spas-1 specific CD8+ T cells. We also observed a similar kinetic of antigen-specific T cells in a metastatic melanoma patient with a low tumor burden who was treated with combined CTLA-4 and PD-1 blockade. Using MHC/peptide multimers, we detected a transient increase in MART-1-specific CD8+ T cells after two cycles of combination treatment, which was subsequently lost at later time points (FIG. 3I and FIG. 10). This paralleled with the patient's clinical course, where the patient had a partial response after two cycles of combination treatment, but then subsequently progressed.

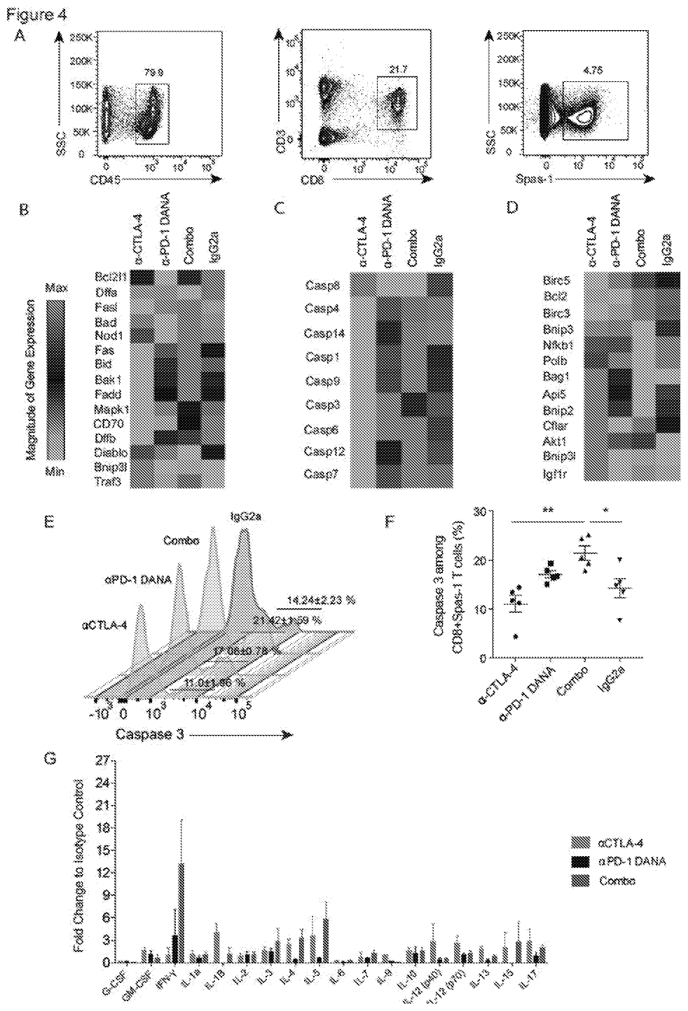

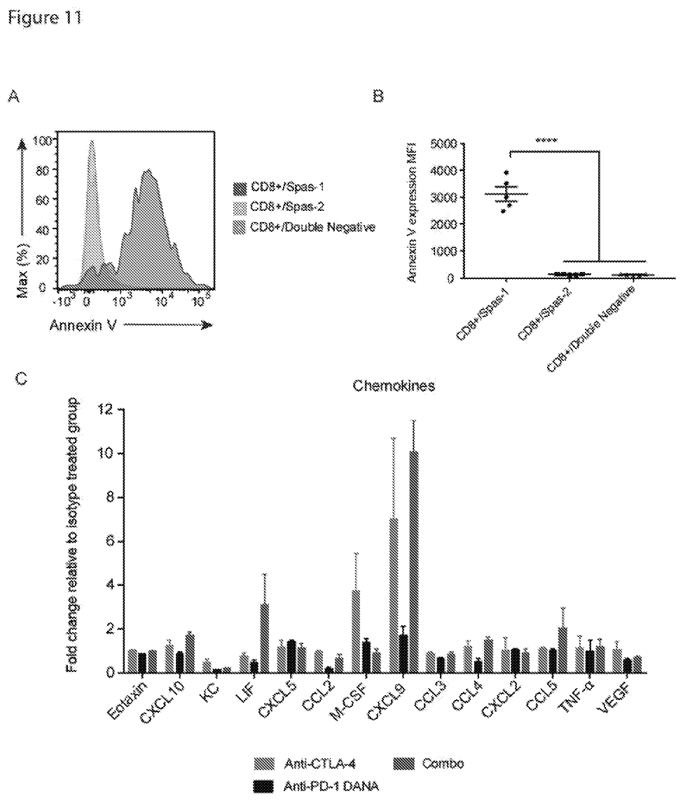

To investigate the loss of antigen-specific T cells, we sorted Spas-1 specific CD8+ T cells from draining lymph nodes of checkpoint inhibitors treated TRAMP-C2 tumor-bearing mice at Day 28 (FIG. 4A). RNA was then extracted from sorted cells, and cDNA generated and analyzed by gene expression. Spas-1 specific CD8+ T cells isolated from the anti-CTLA-4 monotherapy group had low gene transcription levels in the pro-apoptotic gene clusters (FIG. 4B) and Caspase family (FIG. 4C). In contrast, the combination group had increased expression levels of pro-apoptotic genes and Caspase. Consistent with these findings, anti-CTLA-4 treatment alone was associated with higher expression of anti-apoptotic genes compared to the combination treatment group (FIG. 4D). Flow cytometry analysis confirmed that the combo treatment induced higher levels of active cleaved-Caspase-3 in Spas-1 specific CD8+ T cells compared to anti-CTLA-4 alone (FIGS. 4E-F; 21.42±1.59 versus 11.0±1.96; P<0.01). Interestingly, the Spas-1 reactive CD8+ T cells were more susceptible to treatment-induced apoptosis compared to minor Spas-2 epitope-reactive T cells (FIGS. 11A-B; P<0.0001). Overall, these results indicate that the combination of anti-CTLA-4 and anti-PD-1 DANA treatment during early stage of tumor growth can promote apoptosis of antigen specific T cells, particularly those recognizing the immunodominant tumor epitope.

### IFN-γ Promotes Apoptosis of Activated Antigen-Specific T Cells

PD-1 blockade has been shown to prevent terminal exhaustion of antigen-specific T cells rather than promote apoptosis. We hypothesized that the contraction of antigen-specific T cells observed could result from cytokines mediating T cell contraction (T. Yajima et al., J Immunol 176, 507-515 (2006)) and homeostasis (C. D. Surh, J. Sprent, Immunity 29, 848-862 (2008)). We first investigated changes in cytokine levels after treatment during early tumor development. Analysis of serum samples two days after the last dose of checkpoint antibodies showed that IFN-γ, IL-5 and IL-15 were increased in the combination group compared to the other treatment groups (FIG. 4G). In addition, the chemokine ligands CXCL9, LIF and CCL5 were also increased after combination treatment (FIG. 11C). Among the three cytokines, IFN-γ has been shown to mediate the contraction of antigen specific CD8+ T cells (K. Tewari, Y. Nakayama, M. Suresh, J Immunol 179, 2115-2125 (2007)), induce T cell apoptosis (Y. Refaeli et al., The Journal of experimental medicine 196, 999-1005 (2002)) and increase the expression of CXCL9, LIF and CCLS (P. Guimalda et al., Oncoimmunology 2, e25752 (2013); X. Wen et al., Journal of interferon & cytokine research: the official journal of the International Society for Interferon and Cytokine Research 30, 653-660 (2010)).

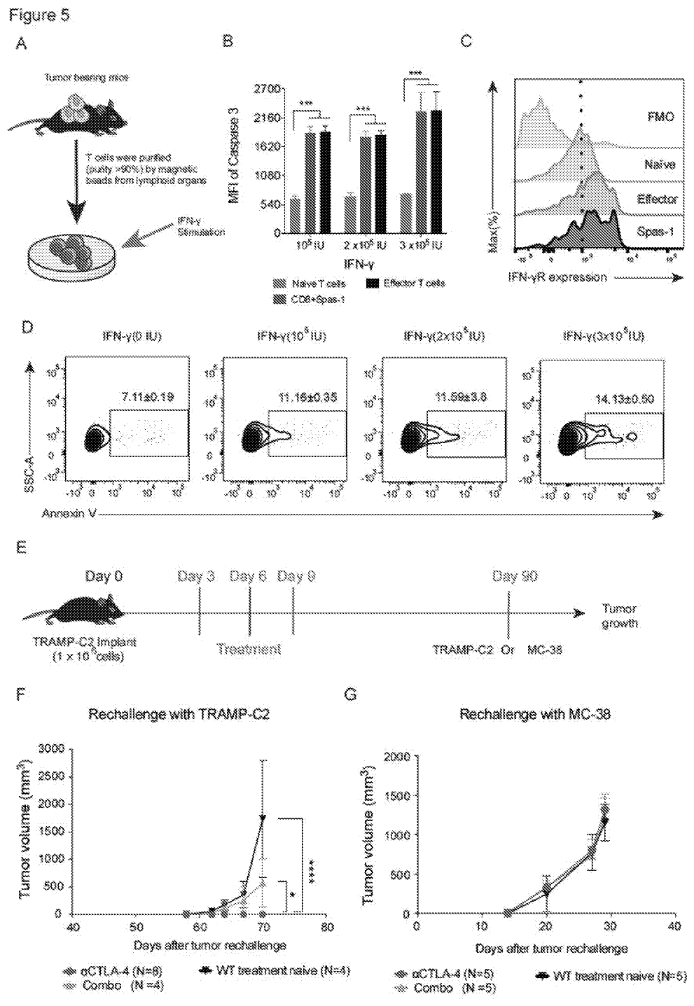

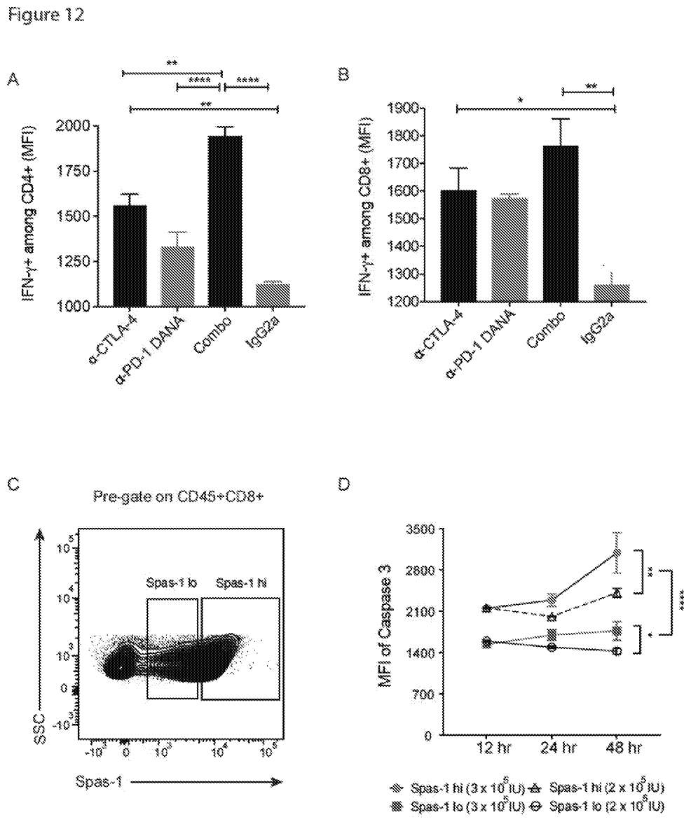

We hypothesized that the loss of Spas-1 specific CD8+ T cells may be related to increased IFN-γ signaling. We first investigated T cells subsets two days after antibody treatment, and found that CD4+ T cells from combination-treated mice secreted higher amounts of IFN-γ compared to monotherapy or isotype (FIG. 12A). A similar pattern was seen in the CD8+ T cells in combination group when compared to isotype (FIG. 12B; P<0.01), but no difference was observed compared to monotherapy. To further evaluate the effects of IFN-γ on antigen specific T cells, T cells were purified from spleens of tumor-bearing mice and stimulated with different concentrations of recombinant IFN-γ in vitro (FIG. 5A). 72 hours after the stimulation, the level of active Caspase-3 expression was examined among T cells subsets, including naïvee T cells (CD45+CD8+CD44-CD62L+), antigen specific T cells (CD45+CD8+Spas-1), and effector T cells (CD45+CD8+CD44+CD62L-). Activated effector T cells and antigen specific T cells were more susceptible to IFN-γ induced active Caspase-3 expression compared to naïvee T cells (FIG. 5B; 2297.33±305.84 versus 2281.67±305.89 versus 723±17.54; P<0.001). In addition, effector T cells and CD8+ Spas-1 T cells showed higher levels of IFN-γ receptor expression compared to naïvee T cells (FIG. 5C).

It has been shown that T cell homeostasis can be achieved by down-modulation of antigen specific T cell receptor (TCR) signaling (A. M. Gallegos et al., Nature immunology 17, 379-386 (2016)). To investigate whether combination treatment eliminated different T cell clones depended on the strength of TCR binding to cognate cancer epitope, we gated on CD8 subsets and investigated Spas-1hi versus Spas-11o CD8+ T cell clones (FIG. 12C). Flow cytometry analysis showed that Spas-1hi T cells were more susceptible to Caspase-3 expression compared to Spas-11o T cells upon IFN-γ induction (FIG. 12D; P<0.0001). Finally, peripheral blood mononuclear cells from a melanoma patient that received nivolumab immunotherapy were isolated and cultured in vitro with different concentrations of IFN-γ stimulation. We observed a gradual increase in apoptosis corresponding to IFN-γconcentration in CD8+ T cells (FIG. 5D). Taken together, these data show that combination treatment leads to a drastic rise in IFN-γ levels, which can induce the apoptosis of tumor-specific T cells.

### Loss of Antigen-Specific T Cells Negatively Impacts Anti-Tumor Memory Responses.

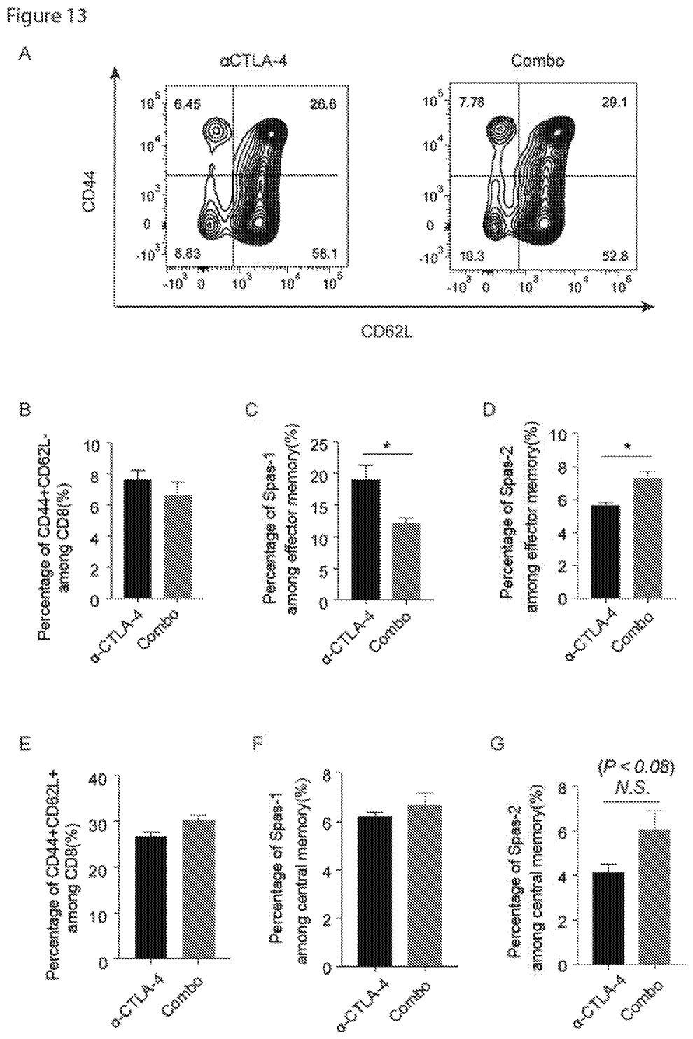

The generation of long-term T cell memory responses is important for an effective and durable anti-tumor response. Because the persistence of antigen specific T cells is important in the formation of memory responses (C. A. Klebanoff, L. Gattinoni, N. P. Restifo, Immunological reviews 211, 214-224 (2006)), we evaluated the effect of combination treatment during early tumor growth on memory response formation. Mice challenged with TRAMP-C2 were treated and observed for three months. 20-30% of mice treated with combination treatment and 80-90% of mice treated with anti-CTLA-4 alone were tumor-free at 90 days. These protected mice were rechallenged with either TRAMP-C2 or MC-38 (control) in the contralateral flank (FIG. 5E). Aged control WT mice without prior tumor challenge were used as controls to evaluate primary responses to these tumors. Mice that received prior combination treatment had compromised protection from TRAMP-C2 tumor rechallenge compared to mice previously treated with anti-CTLA-4 alone (FIG. 5F; P<0.05). No differences in tumor outgrowth were observed in MC-38 challenged mice (FIG. 5G), indicating that TRAMP-C2 tumor control observed was mediated by tumor-specific memory responses. To investigate the compromised memory responses, mice were challenged with TRAMP-C2 cell lines, treated with anti-CTLA-4 alone or combination treatment, and tumor-draining lymph nodes were harvested on day 28 (FIG. 13A). There was a decrease in Spas-1 specific CD8+ effector memory T cells (CD45+CD3+CD8+CD44+CD62L-Spas-1) in the combination group compared to anti-CTLA-4 alone (FIG. 13C; 12.23±0.68 versus 19±1.63; P<0.05). Interestingly, Spas-2 specific CD8+ effector memory cells were increased (FIG. 13D; P<0.05). There were no significant changes in the total number of Spas-1-reactive central memory CD8 T cells (CD44+CD62L+Spas-1; FIG. 13F). These data indicate that the loss of Spas-1 specific CD8+ T cells in the combination treatment group also impairs memory responses.

Deficiency of the IFN-γ Receptor in Immune Cells Rescues Anti-Tumor Activity after Combination Therapy

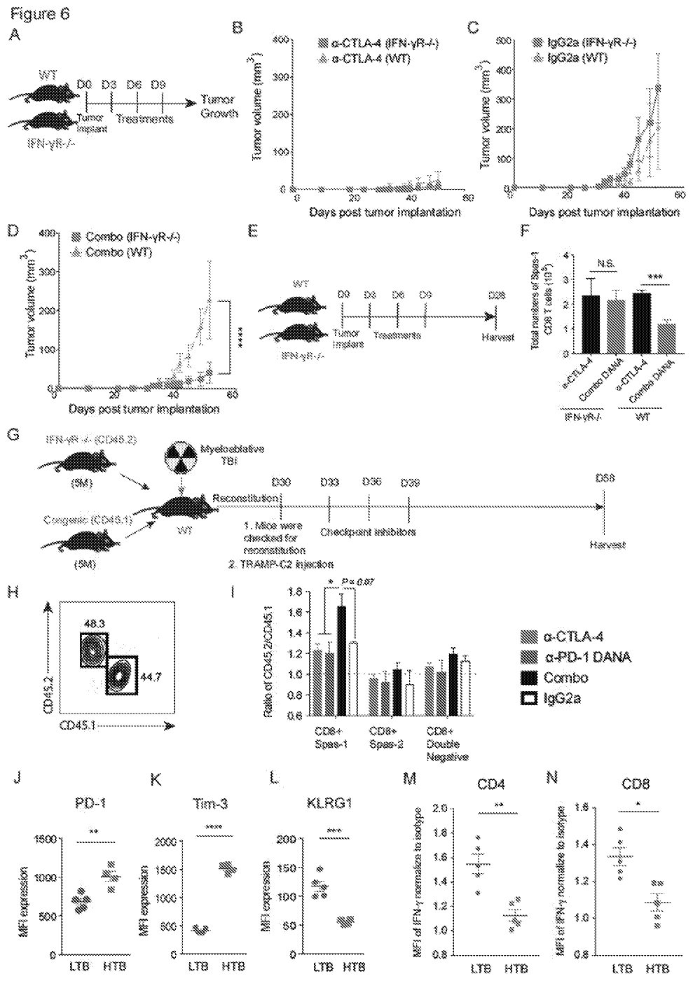

IFN-γ is essential in triggering potent anti-tumor responses by inducing MHC I expression and enhancing antigen presenting capabilities (H. Ikeda, L. J. Old, R. D. Schreiber, Cytokine & growth factor reviews 13, 95-109 (2002)). Although neutralization of IFN-γ can potentially prevent antigen specific T cells loss, neutralization may also abrogate anti-tumor responses. To evaluate whether IFN-γ signaling is important for antigen-specific T cells, we used IFN-γ receptor knockout (RKO) mice (FIG. 6A). T cells from RKO mice can secrete IFN-γ, but cannot respond to the cytokine as they lack corresponding IFN-γ receptors. Mice were challenged with wild type TRAMP-C2 with intact IFN-γ receptors to avoid potential effects from tumor mediated adaptive resistance (S. Spranger et al., Sci Transl Med 5, 200ra116 (2013)). After tumor challenge, there was no difference in tumor growth between WT and RKO mice treated with anti-CTLA-4 (FIG. 6B) or isotype (FIG. 6C). In contrast, combination treatment led to significantly improved anti-tumor efficacy in RKO mice compared to WT mice (FIG. 6D; P<0.0001). To investigate the number of Spas-1-specific CD8+ T cells between RKO and WT mice, spleens were harvested from mice on day 28 (FIG. 6E). In WT mice, the total numbers of Spas-1 specific CD8+ T cells were significantly reduced after combination treatment compared to anti-CTLA-4 alone (FIG. 6F; P<0.001). However, in RKO mice, there was no difference in the total numbers of Spas-1 specific CD8+ T cells (FIG. 6F).

To determine whether IFN-γ signaling in immune cells or non-immune cells (e.g. stromal cells) are responsible for this difference, we performed experiments in bone marrow chimera mice. WT mice underwent myeloablative conditioning and were adoptively reconstituted with bone marrow cells from CD45.2 RKO mice and CD45.1 congenic mice in a 1:1 ratio (FIG. 6G). Hematopoietic recovery and chimerism were checked 30 days after bone marrow transplant to ensure that CD45.2+ and CD45.1+ populations were close to 1:1 in ratio (FIG. 6H). Chimera mice were subsequently implanted with TRAMP-C2 cells on day 30 and treated with three doses of respective antibodies on days 33, 36 and 39. Tumor draining lymph nodes were harvested 28 days after tumor implantation, and the ratio of CD45.2+/CD45.1+ cells in different antigen specific T cell subsets were examined We found a higher ratio of CD45.2+/CD45.1+ cells in the combination treatment group (FIG. 61), indicating a competitive advantage of Spas-1 specific CD8+ T cells from RKO, but not WT, mice after treatment. In summary, these data indicate that the dampening of tumor control observed with combination treatment during early tumor growth is at least partially reversed by knockout of IFN-γ receptors on immune cells, including antigen-specific T cells.

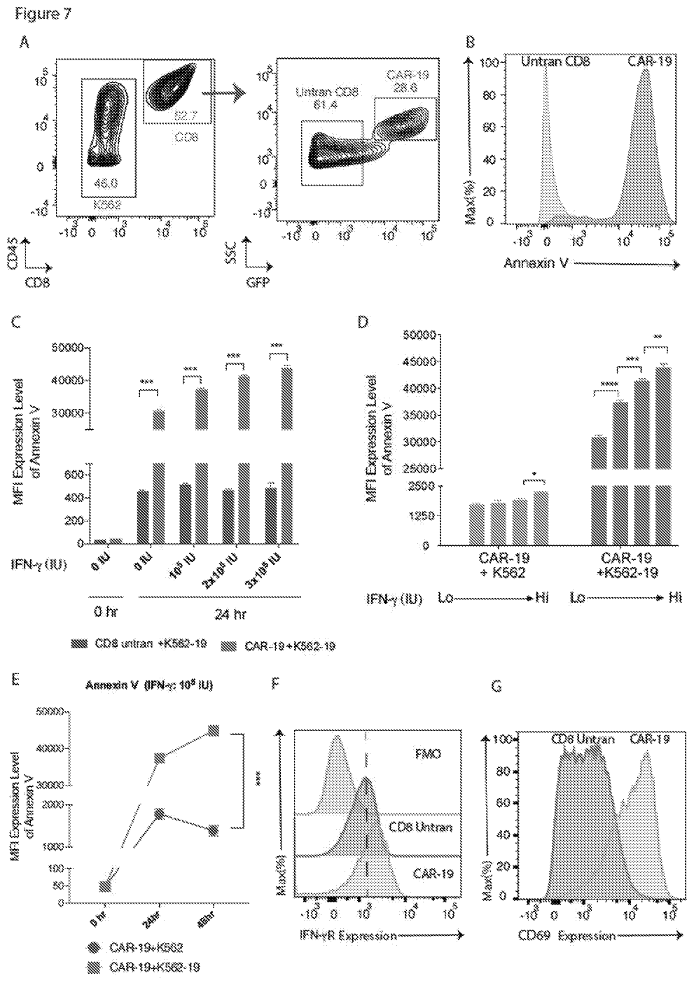

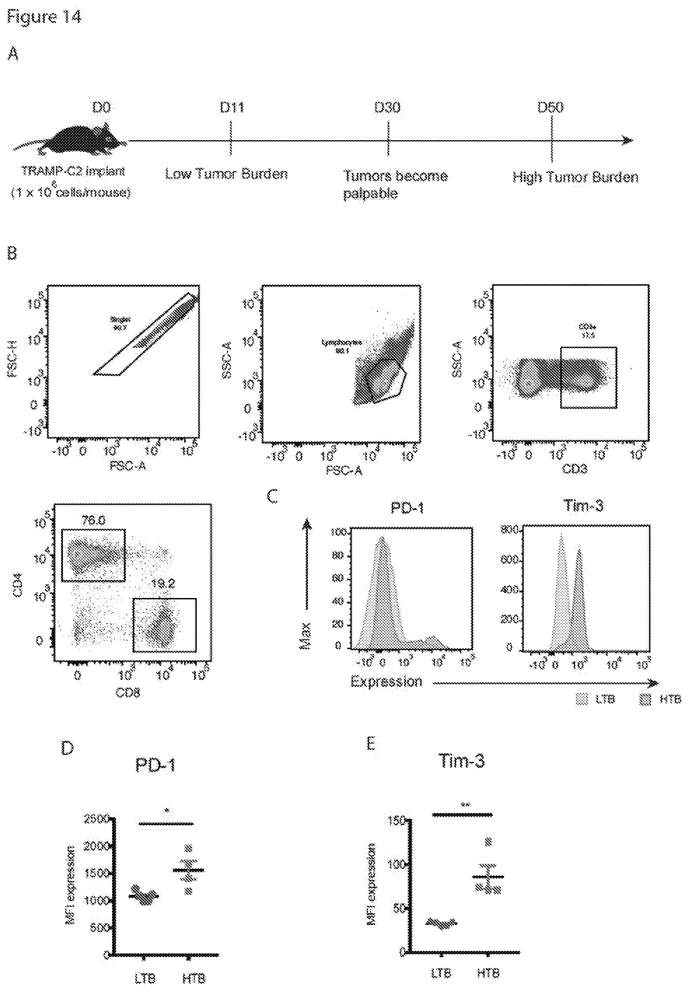

Finally, we sought to investigate the differential effects of checkpoint inhibition in the setting of low tumor burden (LTB) versus high tumor burden (HTB). During tumor development, antigen-specific T cells undergo a dynamic exhaustion process that correlates with tumor progression and tumor burden (A. Schietinger et al., Immunity 45, 389-401 (2016)). To investigate the effects of tumor burden on T cell function, mice were challenged with TRAMP-C2 tumors. T cells were later isolated from spleens on either Day 11 (LTB setting) or Day 50 (HTB setting) post tumor injection (FIG. 14A). We found that both CD4+ and CD8+ T cells isolated from LTB demonstrate significantly lower expression of exhaustion markers with PD-1 (P<0.01) and Tim-3 (P<.0001), but higher expression of KLRG1 (P<0.001) as compared to T cells isolated from HTB (FIG. 14). We then examined whether different degrees of exhaustion status associated with disease burden may impact the outcome of combination treatment. We found that T cells isolated in the combination group demonstrated higher IFN-γ secretion compared to isotype control in both LTB and HTB. However, the amount of IFN-γ was significantly higher in the LTB setting as compared to HTB setting in both CD4+ (FIG. 7M; P<0.01) and CD8+ T cells (FIG. 7N; P<0.05). Overall, our data indicate IFN-γ secretion is blunted as T cells become more exhausted with HTB. Thus, the higher secretion of IFN-γ in mice with LTB induced by combination checkpoint blockade may actually be excessive, leading to apoptosis of tumor-reactive T cells.

In addition, human primary CD8 T cells were isolated from health patients. CD8 T Cells were then transfected with CAR-19 structure to become CAR-19 T cells. Untransfected CD8 T cells were used as control. Both untransfeted CD8 T cells and CAR-19 T cells were cultured in vitro with supplement of 30 IU/ml of hIL-2. For stimulation of T cells, K-562 cancer cells express CD-19 ligand were co-cultured with CAR-19 T cells. See FIGS. 7A-G.

### Discussion

Current cancer immunotherapy strategies aim to counteract the suppressive tumor environment by enhancing antigen recognition of T cell receptors (H. Torikai et al., Blood 119, 5697-5705 (2012)), increasing anti-tumor cytotoxicity capabilities via cytokines (C. A. Klebanoff et al., Proceedings of the National Academy of Sciences of the United States of America 101, 1969-1974 (2004); S. A. Rosenberg, J Immunol 192, 5451-5458 (2014)), or unleashing the “brakes” in the immune system and preventing terminal T cell exhaustion by blocking different immune checkpoint inhibitors (A. Schietinger et al., Immunity 45, 389-401 (2016); P. Sharma, J. P. Allison, Cell 161, 205-214 (2015)). The clinical success of CTLA-4 and PD-1 blockade in melanoma (J. Larkin et al., The New England journal of medicine 373, 23-34 (2015)) has shown combination immunotherapy to be a viable strategy in improving anti-tumor response. It has been shown that T cells isolated from patients treated with dual checkpoint blockade demonstrated a significant increase in IFN-γ levels compared to pre-treatment samples at baseline (R. Das et al., J Immunol 194, 950-959 (2015)), and various combination therapies to enhance IFN-γ production are the subject of ongoing clinical investigation. We found, however, that potent combination therapy with CTLA-4 and PD-1 blockade can actually be detrimental, and we report here on the negative impact of IFN-γ on anti-tumor immunity.

While combination therapy in mice with established tumors achieved improved tumor control, combination treatment in the context of low tumor burden compromised anti-tumor effects in both mice and metastatic melanoma patients. Mechanistically, we found that combination treatment during early tumor development leads to heightened IFN-γ production, which in turn results in apoptosis of the dominant tumor-specific T cells via AICD. In addition to dampening of anti-tumor response, the loss of antigen specific T cells also negatively impacts long-term T cell memory responses. Overall, our results underscored a new role of IFN-γ signaling in the regulation of anti-tumor responses after immune checkpoint therapies.

IFN-γ has conventionally been demonstrated to have immune stimulatory roles that mediate anti-tumor effects. The secretion of IFN-γ from tumor infiltrating lymphocytes can activate both dendritic cells and macrophages to enhance antigen presentation (A. J. Minn, Trends in immunology 36, 725-737 (2015)). IFN-γ signaling on cancer cells can also activate MHC I expression and Stat-1—dependent genes, including P21 and cyclin kinase that inhibit cell cycle progression, resulting in apoptosis of tumor cells (H. Ikeda, L. J. Old, R. D. Schreiber, Cytokine & growth factor reviews 13, 95-109 (2002); J. Gao et al., Cell 167, 397-404 e399 (2016)). However, there is also evidence showing the paradoxical role of IFN-γ in cancer immunotherapies, in particular, its association with acquired resistance (M. R. Zaidi, G. Merlino, Clinical cancer research: an official journal of the American Association for Cancer Research 17, 6118-6124 (2011)). IFN-γ has been shown to promote therapy resistance to immune checkpoint blockade by upregulation of IDO and PD-L1 (S. Spranger et al., Sci Transl Med 5, 200ra116 (2013)) and other co-inhibitory receptors, including Tim-3 and Lag-3 (J. L. Benci et al., Cell 167, 1540-1554 e1512 (2016); S. Koyama et al., Nature communications 7, 10501 (2016)). Here, we demonstrated that IFN-γ signaling can be immunosuppressive, mediating therapy resistance through a PD-L1 independent pathway. Induction of IFN-γ secretion following dual blockade treatments can promote apoptosis of tumor-reactive CD8+ T cells while limiting the formation of effector memory anti-tumor responses. We also found that T cells isolated from melanoma patients respond to IFN-γ induced apoptosis. Overall, our study highlights the importance of type II interferon that not only accounts for cytotoxic effects against cancer cells, but can also mediate the loss of tumor-specific T cells. These results provide a potential mechanism that underlies accelerated tumor growth seen clinically in some checkpoint inhibitor treated patients (S. Champiat et al., Clinical cancer research: an official journal of the American Association for Cancer Research 23, 1920-1928 (2017)), as well as explain why more frequent dosing of combination checkpoint inhibitors is associated with a lower overall response rate in lung cancer patients (M. D. Hellmann et al., The lancet oncology 18, 31-41 (2017)).

Taken together, these results indicate that there exists a potential window within which the immune system can optimally respond to cancer. In the setting of low tumor burden, optimal immunotherapy, such as CTLA-4 or PD-1 blockade alone, can induce invigoration of T cells and provide substantial benefits to cancer patients (A. C. Huang et al., Nature 545, 60-65 (2017)). The blunted IFN-γ responses in high tumor burden create a different threshold where combination checkpoint blockade may be necessary for therapeutic benefit.

SUPPLEMENTAL TABLE 1

| | | Dosage (mg per kg/ | |
|---|---|---|---|
| Name | Clone | injection mouse) | Company |
| Anti-CTLA-4 | UC-10 | 2.5-10 | AbbVie |
| Anti-PD-1 | 17D2 | 10 | AbbVie |
| Anti-PD-1 | RMP1-14 | 10 | Bioxell |
| Anti-PD-1 DANA | 17D2 | 2.5-10 | AbbVie |

Note:
1. Bioxell anti-PD-1 was used in FIG. 3D for comparison. All other anti-PD-1/anti-PD-1 DANA monoclonal antibodies used in this manuscript were provided by AbbVie.

SUPPLEMENTAL TABLE 2

| Markers | Company | Cat No. | Clone | Florescence | Note |
|---|---|---|---|---|---|
| CD3 | Biolegend | 100232 | 17A2 | BV785 | |
| CD4 | Biolegend | 100447 | GK1.5 | BV711 | |
| CD8 | BD Bioscience | 552877 | 53-6.7 | PE-Cy7 | |
| CD16/32 | TONBO | 70-0161-U500 | 2.4G2 | N/A | Fc block |
| CD44 | Biolegend | 103049 | IM7 | BV650 | |
| CD45 | Biolegend | 103126 | 30-F11 | PB | |
| CD45.1 | Biolegend | 110737 | A20 | BV605 | |
| CD45.2 | Biolegend | 109847 | 104 | BV711 | |
| CD62L | Biolegend | 104433 | MEL-14 | BV570 | |
| Foxp3 | eBioscience | 11-5773-82 | FJK-16s | FITC | |
| rIgG2a/k | eBioscience | 11-4321 | eBR2a | FITC | Isotype |
| PD-1 | Biolegend | 135213 | 29F.1Al2 | FITC | |
| rIgG2a/k | Biolegend | 400505 | RTK2758 | FITC | Isotype |
| Tim-3 | Biolegend | 134003 | B8.2C12 | PE | |
| rIgGl/k | Biolegend | 400407 | RTK2071 | PE | Isotype |
| KLRG1 | Biolegend | 138412 | 2F1 | APC | |
| Hamster | Biolegend | 402012 | N/A | APC | Isotype |
| IgG | | | | | |
| IFN-γ | BD Bioscience | 554411 | XMG1.2 | FITC | |
| Rat IgG1 | BD Bioscience | 553924 | R3-34 | FITC | Isotype |
| IFN-γ | BD Bioscience | 740897 | GR20 | BV786 | |
| receptor | | | | | |
| Rat IgG2a | BD Bioscience | 563335 | R35-95 | BV786 | Isotype |
| Caspase-3 | BD Bioscience | 51-68654X | N/A | FITC | Apoptosis |
| Annexin V | BD Bioscience | 51-65874X | N/A | FITC | Apoptosis |
| Spas-1 | NIH tetramer Core | N/A | N/A | PE | Sequence: |
| | | | | | STHVNHL |
| | | | | | HC |
| | | | | | H-2D(b) |
| Spas-2 | NIH tetramer Core | N/A | N/A | APC | Sequence: |
| | | | | | IIITFNDL |
| | | | | | H-2K(b) |

### Materials and Methods

### Supplemental Materials and Methods

### Mice

8-10 week-old aged control male C57BL/6j, Ifngr KO and CD45.1 congenic (C57BL/6j background) mice were obtained from Jackson Laboratory and used in the experiments. Mice were implanted subcutaneously (S.C.) with either TRAMP-C2 or MC-38 cell line at a dosage of 1×10⁶ per mouse at the right flank on day 0, and were treated with different antibodies intraperitoneally (I.P.) on day 3, 6, and 9. In the late intervention TRAMP-C2 group, 1×10⁶ TRAMP-C2 cells were similarly implanted S.C. at the right flank on day 0, but allowed to grow for 30-45 days prior to treatment. Mice with tumor volumes within 50-200 mm³ were selected into different treatment groups before treatment. The average tumor sizes among different treatment groups were checked and ensured to be similar before treatment. Mice were injected with different antibodies I.P. on day 3, 6, and 9. In memory re-challenge experiments, mice were implanted with TRAMP-C2 tumors at a dose of 1×10⁶ per mouse at the right flank on day 0. Mice were then treated with different immune checkpoint antibodies on day 3, 6, and 9. Tumors were measured twice a week, every 3-4 days. Ninety days after the initial tumor implantations (day 90), tumor-free mice from either anti-CTLA-4 or anti-CTLA-4 and anti-PD-1 DANA combination treatment groups were rechallenged with TRAMP-C2 tumors at the left flank at a dosage of 1×10⁶. There were no tumor-free mice treated with anti-PD-1 DANA antibody alone or isotype control. Sibling WT mice without prior tumor challenge or treatment were aged together in the same vivarium and used later as controls for rechallenge experiments. Tumor measurement=L (length)×W (width)×W/2 (mm³); whereas the longer diameter was defined as length and the shorted diameter was defined as width. All mice were maintained at UCSF vivarium in accordance with Institutional Animal Care and Use Committee (IACUC) standards.

### Generation of Chimera Mice

8-10 week-old C57BL/6j mice (H2^{b}) were used as recipient mice and underwent lethal total body irradiation (1050 cGy;¹³⁷Cs source) followed by transplantation from donor CD45.2 Ifngr KO mice and CD45.1 congenic mice. T cell-replete bone marrows were mixed in 1:1 ratio (5×10⁵ cells total) and injected intravenously (I.V.) through the tail vein per recipient mouse. Chimera mice were reconstituted for 30 days and checked for chimerism by tail bleeding. Chimera mice were implanted subcutaneously with 1×10⁶ TRAMP-C2 cells at the right flanks on day 30. Mice were subsequently injected with different checkpoint inhibitors (10 mg/kg/injection/mouse) on day 33, 36, 39. On day 58, mice were sacrificed and cells were harvested from tumor draining lymph nodes. The ratio of CD45.2+/CD45.1+ cells in CD8+Spas1 cells was calculated by dividing the total number of CD45.2+CD8+Spas-1 cells by the number of CD45.1+CD8+Spas-1 cells. The proportion of CD8+Spas-2 and CD8+ double negative subsets were similarly derived. All mice were maintained at the UCSF vivarium in accordance with IACUC standards.

### Real-Time RT-PCR Gene Cytokine Arrays

CD45+CD3+CD8+Spas-1 T cells were sorted from draining lymph nodes from TRAMP-C2 bearing mice on day 28 after treatment. RNA was extracted from sorted CD8+Spas-1 T cells using an Ambion micro RNA isolation kit (AM1931) according to the manufacturer's protocol, and genomic DNA was eliminated using DNase kit purchased from Qiagen. RNA quality was checked by the A260/A280 ratio using NanoDrop Lite (Thermal Scientific). lOng RNA from each sample was used for subsequent cDNA synthesis. cDNA was synthesized and cDNA templates pre-amplified were using RT2 PreAMP cDNA synthesis kit (Qiagen Cat 330451) according the manufacturer's protocol and ProFlex PCR machine (Applied Biosystems). cDNA samples derived were evaluated for apoptotic gene expression arrays using RT² Profiler™ PCR Array kits purchased from Qiagen (PAMM-012Zc-12, Cat 330231) with SYBR Green qPCR Mastermix (Qiagen Cat 330522). Quantitative real-time RT-PCR arrays was performed using Applied Biosystems Cycler (AB Step-ONE Plus). PCR arrays were analyzed and gene expression heat map generated using software provided on Qiagen website under Data Analysis Center. All samples passed quality control (QC). Expression level for each gene is presented as fold change in comparison to internal control of house keeping genes (beta-actin, Gus and Hsp90ab1) in each group. Array data are available in GEO database under accession number: GSE95433

Clinical Outcomes with Immune Checkpoint Inhibition

Patients were treated with either PD-1 monotherapy with pembrolizumab 2 mg/kg or 10 mg/kg (n=101), or PD-1/CTLA-4 combination therapy with ipilimumab 3 mg/kg plus nivolumab 1 mg/kg (n=51). Best objective response rate was determined by RECIST 1.1. Baseline tumor size was calculated by summing the largest diameter of the target lesions per RECIST 1.1. Patients were stratified according to the baseline tumor size into ≤6 cm, >6-≤11 cm, or >11 cm. Patients with a complete or partial response were categorized as responders, and those with stable disease or progressive disease as their best response were categorized as non-responders. The responder fraction was calculated by dividing responders/all patients. The error bars represent SEM. Significance was calculated by the Mann-Whitney test, p value is 2 sided, NS, p<0.05.

### MHC/Peptide Multimer Staining

Cryopreserved PBMCs were collected from an HLA-A*0201 patient with metastatic melanoma receiving 4 cycles of ipilimumab and nivolumab followed by nivolumab monotherapy maintenance. PBMCs were thawed and counted by Vi-Cell to determine cell numbers and stained with HLA-A*0201 MHC/peptide dextramers specific for different melanoma antigens (Melanoma Dextramer Collection 1, Cat. No. RX01; Immudex). The dilution rate and staining procedures followed manufacturer's protocols. Flow samples were run on a BD X-50 and analyzed by Flow-Jo software. The clinical trial were approved and under supervision of IRB at University of California, San Francisco.

### Antibodies Generation

Antibodies against mouse PD-1 were generated by immunizing HSD rats with recombinant mouse PD-1 protein (R&D Cat: 1021-PD). Hybridomas were generated by fusing IgG producing cells from immunized mice with myeloma cells (NS0-Mouse Myeloma, PTA-4796), and screened for binding to PD-1. The UC10-4F10-11 hybridoma expressing mouse anti-CTLA-4 antibody was purchased from ATCC (HB-304). The antibody variable domains were cloned from the hybridomas and expressed as murine IgG2a WT or with mutations to inactivate FcR binding (D265A; N297A; DANA) (Baudino, L., et al. (2008). Journal of immunology 181, 6664-6669; Chao, D.T., et al. (2009) Immunological investigations 38, 76-92). PD-1 and CTLA-4 antibodies were additionally screened for neutralization of the PD-1—PD-L1/L2 or CTLA-4—CD86 interactions, respectively.

### Antibodies and GVAX

Anti-CTLA-4, anti-PD-1, anti-PD-1 DANA and IgG2a antibodies were obtained from Abbvie or Bioxell (Supplemental Table 1). All antibodies were stored in −80° C. in small working aliquots to avoid repeated freeze-thaw cycles before use. Antibodies were dissolved in phosphate buffer saline (PBS) and injected I.P. at indicated time points. For combination treatments with anti-CTLA-4 and GVAX, 10⁶ irradiated (10,000 rads) GVAX cells were injected S.C. into the skin over the neck on day 3, 6 and 9, at the same time as I.P. antibody injection.

In Vitro T Cell Cultures and Stimulation with Different Concentrations of Recombinant IFN-γ

Splenocytes were harvested from TRAMP-C2 bearing C57BL/6j mice on day 50 and T cells were purified by magnetic beads according to the manufacturer's protocol (Miltenyi Biotec Cat 130-095-130). Cells were checked for over 90% purity. Purified T cells were suspend in DMEM (UCSF cell culture core) +10% fetal bovine serum (Lonza Cat 14-501F) +1% penicillin/streptomycin (UCSF cell culture core)+murine 20 IU IL-2 (Peprotech Cat 212-12), and seeded in 96 wells at 2×10⁵ cells per well. Recombinant murine IFN-γ (Peprotech Cat 212-12) was added into the wells at indicated concentrations. Cells were cultured for 12-72 hours and analyzed by flow cytometry. For the patient sample, peripheral blood mononuclear cells (PBMCs) were isolated by Ficoll (Sigma Cat F4375) and PBMCs were seeded in 96 wells at 2×10⁵ cells per well supplemented with 20 IU human IL-2 (Peprotech Cat 200-02). Recombinant human IFN-γ (Peprotech Cat 212-12) was added to the wells. Cells were harvested and analyzed by flow cytometry 72 hours after incubation.

### Cell Culture

Tumor cell lines, TRAMP-C2 and MC-38, were cultured for cell injection into C57BL/6j male mice. TRAMP-C2 cell medium composed of DMEM (UCSF cell culture core), 5% fetal bovine serum (FBS; Lonza Cat. 14-501F), 5% Nu-serum IV (Corning Cat. 355504), 0.005 mg/ml bovine insulin (Sigma Cat. 10516), 10nM dehydroisoandrosterone (Sigma Cat. D5297), and 1% penicillin/streptomycin (UCSF cell culture core). MC-38 medium composed of DMEM (UCSF cell culture core), 5% fetal bovine serum (Lonza Cat. 14-501F), 5% Nu-serum IV (Corning Cat. 355504), and 1% penicillin/streptomycin (UCSF cell culture core). Frozen cell lines were thawed in the water bath at 37° C. before transfer into corresponding pre-warmed media. After wash, cells were then pelleted and resuspended in fresh media before passage into culture flasks. Once every two days, culture flasks were checked for confluence with a light microscope. Before cells overcrowd the culture flask (>90% of confluency), old media from the flask was decanted into waste and 10 mL of PBS added as a rinse. After cells were rinsed with PBS, the solution was removed and 5 mL of 0.05% trypsin with EDTA (UCSF cell culture core) was introduced into the flask. Subsequently the flask was placed in a CO2 incubator at 37° C. for 5 minutes for trypsinization of adherent cells. After incubation, the trypsin was neutralized with plentiful media, pelleted, and then resuspended in new media before a fractional transfer into new culture flasks. For cell injection into mice, instead of the fractional transfer step, cells were washed and pelleted with PBS twice to remove the presence of FBS. Prior to injection, cells were adjusted with PBS to a concentration of 10⁷ cells per mL with each needle containing 1×10⁶ cells in 100 uL.

### Mouse Serum Cytokine Arrays

Two days after the final checkpoint inhibitor treatment, mouse sera were collected and sent to Eve Technologies (Calgary, Alberta, Canada) for analysis with mouse cytokine 31-plex discovery assay (Cat No: MD31). Serum cytokine levels from treatment groups were each divided by the serum cytokine level of the IgG2a control group to calculate as fold changes. These fold changes were graphed with the Prism 7 software.

### Tissue Preparations

Spleens were surgical removed with sterilized surgical equipment and crushed with the blunt end of a 10 mL syringe on petri dishes containing 5 mL of PBS. The spleen mixtures were separately filtered through a 70 μM filter into a 50 mL conical tube, centrifuged at 1500rpm for 5 minutes at 4° C. After wash, cell pellets were resuspended in 5 mL of red blood cell lysis solution (Santa Cruz Biotechnology; Cat sc-296258) on ice for 5 minutes and stopped with the addition of 30 mL of PBS. After wash, cells were reconstituted for counting by Vi-Cell (Beckman Coulter, U.S.A.). Draining lymph nodes were extracted with sterilized surgical equipment and crushed between the frosted surfaces of super-frosted microscope slides into wells containing PBS. Cell mixtures were then filtered through a 70 μM filter into 15 mL conical tubes. Cells were then washed and counted. Tumors were removed from mice with sterile surgical instruments followed by sectioning for paraformaldehyde fixation or flow cytometry analysis. Tumor tissues for flow analysis were kept moist with 1 mL collagenase IV digest media (DMEM+10%FCS+1% penicillin/streptomycin+Collagenase IV+DNase) and minced with scalpel blades. Tumor cell mixtures were then transferred into 15 mL conical tubes and filled with additional 9 mL of collagenase digest media. Tumor samples were subsequently placed on a 37° C. shaker for 1 hour. Samples were filtered through a 100 μM filter into a 50 mL conical tube and washed with PBS before centrifugation. Finally, tumor cell pellets were resuspended and counted before subsequent flow staining.

### Antibodies and Flow Staining

The flow cytometry protocols were previously described (Sckisel et al., 2015). Single cell suspensions (1 million cells) were first incubated with Fc Block (BD Pharmingen. San Diego, Calif.) for 10 minutes, then co-incubated with antibodies for 20 minutes at 4° C. followed by washing with staining buffer (PBS +1% FBS). Foxp3 and intracellular staining were performed using eBioscience intracellular kit (Cat#00-5523-00) according to the manufacturer's protocol. Active Caspase-3 staining was performed by using BD Caspase-3 apoptosis kit (BD Cat 550480), and Annexin V staining was performed using BD Annexin V apoptosis detection kit (BD Cat 556547) according to the manufacturer's protocol. Flow cytometry was performed on Fortessa X20 Dual, and data analyzed by FlowJo software (TreeStar). Details on flow cytometry antibodies used in this study can be found in Supplemental Table 2.

### Histology and Immunohistochemistry

Tissues harvested from mice were placed in 4% formalin, followed by 70% alcohol and PBS before embedding. Tissues were then embedded in paraffin, sectioned, and stained with hematoxylin and eosin. Tissue sections were evaluated by a board certified pathologist (M.C.). Images were visualized using an Olympus Vanox AHBS3 microscope with an Olympus SPlan Apo x 20/0.70 NA objective (Olympus, Woodbury, N.Y.). A diagnostic instrument spot RT color digital camera utilizing Spot software version 4.0.2 was used to acquire the images (Diagnostic Instruments, Sterling Heights, Mich.) Immunohistochemistry were performed as previous described (DuPage, M., et al, (2011). Cancer cell 19, 72-85). Tumor tissues were fixed in 4% paraformaldehyde, processed, embedded in paraffin, then cut into 5 μm sections. Paraffin sections were blocked with 3% hydrogen peroxide solution (Sigma Cat H1009), vector streptavidin/biotin (Vector Laboratories cat. SP-2002), and CAS-Block protein block (ThermoFisher Cat. 008120), then stained with CD8 antibody (Biorbyt Cat orb10325).

### Statistics.

Data shown in this manuscript were presented as mean±SE. Tumor growth curves at different time points were plotted by using Prism 7 and analyzed by two-way ANOVA with a Tukey post hoc test comparison among groups. Flow cytometry data were analyzed by one-way ANOVA with a Tukey post hoc test. P-values less than 0.05 were considered statistically significant. Gene expression arrays were analyzed by software provided by Qiagen website under Data Analysis Center.

### Example 2

C57B1/6j mice were implanted with TRAMP-C2 tumors (10⁶ cells per mouse) on day 0 and treated with combination therapies (anti-CTLA-4+anti-PD-1 DANA) at day 3, 6, and 9. For group 3 and group 4, mice were additionally treated with a JAK inhibitor twice a week from day 11 for total five doses. Table 1 demonstrated the tumor incidence rate at day 55.

TABLE 1
Tumor incidence rate (%)

| | | Mice have | Tumor incidence |
|---|---|---|---|
| Groups | Treatments | tumors | rate (%) |
| 1 | Isotype | 6/6 | 100 |
| 2 | Combo | 6/7 | 85.7 |
| 3 | Combo + JAKi | 3/7 | 42.8 |
| 4 | JAKi | 6/6 | 100 |

As shown above, the combination of a JAK inhibitor with anti-PD-1 and anti-CTLA-4 therapy resulted in a much lower rate of tumor incidence,

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

1. An human tumor-specific T-cell that does not express a functional Interferon-γ (IFN-γ) receptor.

2. The T-cell of claim 1, wherein the T-cell comprises a mutation compared to wildtype that blocks IFN-γ receptor expression.

3. The T-cell of claim 2, wherein the mutation is a mutation in an IFN-γ receptor promoter or IFN-γ receptor coding sequence.

4. The T-cell of claim 1, wherein part or all of a coding sequence for IFN-γ receptor has been deleted.

5. The T-cell of claim 1, wherein the T-cell comprises an siRNA or antisense polynucleotide that inhibits expression of IFN-γ receptor.

6. The T-cell of claim 1, wherein the T-cell comprises a tumor-specific T-cell receptor.

7. The T-cell of claim 1, wherein the tumor-specific T-cell receptor is heterologous to the T-cell.

8. The T-cell of claim 1, wherein the T-cell is bound by a bispecific binding reagent that binds CD-3 and a tumor antigen.

9. The T-cell of claim 8, wherein the tumor antigen is CD-20.

10. The T-cell of claim 8, wherein the bispecific binding reagent is a bispecific antibody.

11. The T-cell of claim 1, wherein the T-cell comprises a heterologous chimeric antigen receptor (CAR).

12. A method of killing cancer cells in a human, the method compri sing,
administering to the human a sufficient number of the T-cell of claim 1 to kill cancer cells in the human .

13. The method of claim 12, further comprising administering to the human a CTL-4 inhibitor and a PD-1 inhibitor.

14. The method of claim 12, wherein the T-cells have been obtained from the human and then altered to inhibit expression of the functional Interferon-γ (IFN-γ) receptor.

15. The method of claim 12, wherein the human has melanoma.

16. The method of claim 12, further comprising administering to the human a sufficient amount of an antibody that binds to IFN-γ to promote survival of the administered T-cells.

17. A method of killing cancer cells in a human, the method comprising,
administering to the human an effective amount of a JAK inhibitor, a CTL-4 inhibitor and a PD-1 inhibitor, thereby killing cancer cells in the human.

18. The method of claim 17, wherein the human has melanoma.

19. The method of claim 17, further comprising administering to the human a sufficient number of a human tumor-specific T-cell that does not express a functional Interferon-γ (IFN-γ) receptor.
